SEQUENCE LISTING (5 sequences)

SEQ ID NO: 1 - DNA, 191 bp, Homo sapiens
  source 1..191: mol_type = genomic DNA
cccagggggtgacgtgcacttagagaaactcggggaaggcctgggtgtgcgacccctcctctgcggcagcagcgccggggccgactctgaaggcttccatggggaaaggagggtttttcagcaggtggtggcggagcgggaggacgatagggcaagtgtgtgagcagaagcagccagagggctgggtctgt

SEQ ID NO: 2 - DNA, 27 bp, synthetic construct
  source 1..27: mol_type = other DNA
tttagggggtgaagtgtatttagagaa

SEQ ID NO: 3 - DNA, 19 bp, synthetic construct
  source 1..19: mol_type = other DNA
acaaacccaaccctctaac

SEQ ID NO: 4 - DNA, 23 bp, synthetic construct
  source 1..23: mol_type = other DNA
ggtgaagtgtatttagagaaatt

SEQ ID NO: 5 - DNA, 18 bp, synthetic construct
  source 1..18: mol_type = other DNA
ggagggttttttagtagg

(19) United States
(12) Patent Application Publication
(10) Pub. No.: US 2025/0027171 A1
(43) Pub. Date: Jan. 23, 2025

(54) URINARY RAD9 BIOMARKER DIAGNOSTIC AND PROGNOSTIC ASSAY FOR PROSTATE CANCER

(71) Applicant: The Trustees of Columbia University in the City of New York, New York, NY (US)

(72) Inventors: Howard B. LIEBERMAN, Tenafly, NJ (US); Catherine S. SPINA, New York, NY (US); Kevin M. HOPKINS, Leonia, NJ (US)

(21) Appl. No.: 18/908,384

(22) Filed: Oct. 7, 2024

Related U.S. Application Data
(63) Continuation of application No. PCT/US23/65400, filed on Apr. 5, 2023.
(60) Provisional application No. 63/327,437, filed on Apr. 5, 2022.
(60) Provisional application No. 63/340,641, filed on May 11, 2022.

(51) Int. Cl. C12Q 1/6886

(52) U.S. Cl. CPC C12Q 1/6886; C12Q 2600/154

(57) ABSTRACT

Described herein is a method for diagnosing cancer in a subject, the method comprising determining methylation status at a plurality of CpG sites within a region of a RAD9 gene in DNA present in a urine sample from the subject, wherein an amount of methylation of the plurality of CpG sites that exceeds a threshold indicates that the subject has cancer. Also described are methods of treatment, methods of assessing a methylation status of DNA present in a urine sample obtained from a human subject, methods of detecting methylation or unmethylation of a RAD9 DNA molecule of a human subject, kits, nucleic acid primers, and compositions.

This application claims priority to U.S. Provisional Application No. 63/327,437 filed Apr. 5, 2022, and U.S. Provisional Application No. 63/340,641 filed May 11, 2022, the contents of each of which are hereby incorporated by reference in their entirety.

### GOVERNMENT SUPPORT

This invention was made with government support under grants CA130536, TR001873, and CA013696 awarded by the National Institutes of Health. The government has certain rights in the invention.

All patents, patent applications and publications, and non-patent publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### TECHNICAL FIELD

The present invention relates generally to diagnostic and prognostic devices, kits, compositions, and methods for cancer. More particularly, the present invention relates to diagnostic and prognostic urine-based assays, devices, kits, and compositions for cancer using RAD9 as a biomarker.

### BACKGROUND

Prostate cancer is the second most common cancer and third leading cause of cancer-related death in men. Speiser D E, Ho P C, Verdeil G. Regulatory circuits of T cell function in cancer. Nat Rev Immunol. 2016 October; 16(10): pp. 599-611; Litwin M S, Tan H J. The diagnosis and treatment of prostate cancer: A review. JAMA. 2017 Jun. 27; 317(24):2532-2542. One in eight men are diagnosed with prostate cancer during their lifetime translating to approximately 250,000 new diagnoses and 35,000 prostate cancer-related deaths per year. Prostate cancer is screened either through a digital rectal examination or prostate-specific antigen (PSA) blood test. Descotes J L. Diagnosis of prostate cancer. Asian J Urol. 2019 April; 6(2):129-136. Tumor aggressiveness is graded based on prostate biopsy samples or genomic testing. While recommendations vary, men between the ages of 50 to 69 years old may choose to undergo periodic prostate cancer screening by digital rectal exam and serum PSA measurement. PSA has a diagnostic specificity of 90% in all-comers, but only 50% in high-grade subtypes. While serum PSA is an established diagnostic for prostate cancer, its accuracy and sensitivity are poor for the most aggressive forms of the disease: metastatic castrate-resistant prostate cancer and aggressive-variant histologies including neuroendocrine prostate cancer. Although first line therapy for men with metastatic prostate cancer includes hormonal therapy (ADT), the selective pressure of long-term ADT promotes a transformation to aggressive-variant histologies, including neuroendocrine differentiation that fail to express PSA. Hence, PSA falls short as an effective biomarker for men with the most lethal forms of prostate cancer. For localized disease, PSA has a relatively high sensitivity (>70%) and specificity (>90%) as a diagnostic tool. Unfortunately, for men with castrate resistant prostate cancer (CRPC) and aggressive-variant histologies such as neuroendocrine prostate cancer, the disease can behave in an androgen-independent fashion and thus be uncoupled from PSA expression. This results in serum PSA being of limited use as a biomarker for these patients. Hence, the most lethal forms of prostate cancer do not have a reliable biomarker for diagnostic and therapy response-based evaluation and management. As well, PSA can be elevated due to non-cancer related reasons (e.g., sexual activity, prostatitis, bicycle riding) resulting in false positives with a PSA diagnostic test. On the other hand, PSA abundance is not always elevated in cancer patients resulting in false negatives (e.g., neuroendocrine prostate cancer). There exists a need for accurate and reliable non-invasive diagnostic and prognostic assays, devices, kits, and compositions for prostate cancer.

### SUMMARY

In one aspect, described herein is a method of assessing a methylation status of DNA present in a urine sample obtained from a human subject comprising assessing the methylation status at a plurality of methylation sites within one or more regions of a RAD9 gene.

In some embodiments, the assessing comprises performing a bisulfite conversion of the one or more regions of the RAD9 gene, amplifying the converted one or more RAD9 regions or the complement thereof with primers comprising a nucleic acid sequence that is complementary to the converted one or more RAD9 regions under selective hybridization conditions, and detecting the methylation status of one or more cytosines in the one or more RAD9 regions. In some embodiments, the one or more regions of the RAD9 gene comprises a RAD region comprising transcription suppressor domain of intron 2. In some embodiments, detecting the methylation status of the one or more cytosines comprises detecting a methylation status of one or more cytosine residues comprising CpG sites 404 to 518 within intron 2. In some embodiments, the primers comprise SEQ ID NO: 2 and SEQ ID NO: 3. In some embodiments, detecting the methylation status of the one or more cytosines comprises sequencing the amplified converted RAD9 region using primers comprising SEQ ID NO: 4 or SEQ ID NO: 5.

In another aspect, described herein is a method of detecting methylation or unmethylation of a RAD9 DNA molecule of a human subject, the method comprising: (a) reacting an isolated RAD9 DNA molecule from a urine sample of the human subject with a bisulfite salt thereby forming a reacted RAD9 DNA molecule; (b) contacting the reacted RAD9 DNA molecule with a probe or a primer complementary to a sequence at or within 100 nucleotides of a plurality of cytosine methylation sites; and (c) detecting a presence or an absence of uracil in the reacted RAD9 DNA molecule at the plurality of cytosine methylation sites, thereby detecting methylation or unmethylation of the RAD9 DNA molecule of the human subject.

In some embodiments, the method comprises isolating the RAD9 DNA molecule from a urine sample of the human subject. In some embodiments, the plurality of cytosine methylation sites is within a region of the RAD9 gene comprising transcription suppressor domain of intron 2. In some embodiments, the plurality of cytosine methylation sites comprises CpG sites 404 to 518 within intron 2. In some embodiments, the method further comprises determining whether a methylation level of the plurality of cytosine methylation sites is at least about 1.5 times greater or about 1.5 to about 4.5 times greater methylation in DNA molecules isolated from the urine sample of the human subject compared to an amount of methylation at a plurality of the same cytosine methylation sites in isolated RAD9 DNA molecules present in a second urine sample obtained from a healthy subject that does not have cancer or in a cohort of urine samples obtained from a plurality of healthy subjects that do not have cancer, and if the methylation level of the plurality of cytosine methylation sites is at least about 1.5 times greater or about 1.5 to about 4.5 times greater, administering to the human subject a treatment to treat or prevent prostate, bladder, or kidney cancer, wherein the treatment comprises surgery, radiation therapy, chemotherapy, immunotherapy, or administering an active agent comprising antineoplastic properties. In some embodiments, the active agent comprising antineoplastic properties is a PARP inhibitor.

In another aspect, described herein is a kit for detecting a cytosine methylation of a region of a RAD9 gene, the kit comprising: a primer set comprising a plurality of forward and reverse primers designed to amplify one or more partially methylated forms of a DNA form of the RAD9 gene in which unmethylated cytosine residues present in DNA present in a urine sample from a subject have been modified, wherein the primer set includes primers comprising SEQ ID NO: 2 and SEQ ID NO: 3.

In some embodiments, the kit additionally comprises an agent which modifies unmethylated cytosine residues. In some embodiments, the agent is a bisulfite salt. In some embodiments, the kit additionally comprises a plurality of reagents configured to effect DNA amplification and/or detection. In some embodiments, the primer set is directed to detecting methylation at a plurality of CpG sites comprising 404 to 518 within intron 2 of RAD9. In some embodiments, the subject is a human. In some embodiments, the kit additionally comprises a plurality of reagents configured to perform bisulfite sequencing or bisulfite pyrosequencing. In some embodiments, the kit additionally comprises a plurality of reagents configured to isolate an amount of cell-free DNA from the urine sample. In some embodiments, the amount of cell-free DNA isolated by the plurality of reagents in the kit is about 500 μg to about 2 μg. In some embodiments, the amount of cell-free DNA isolated by the plurality of reagents in the kit is about 200 ng to about 500 ng. In some embodiments, the kit additionally comprises a plurality of reagents configured to isolate an amount of cellular DNA from the urine sample. In some embodiments, the amount of cellular DNA isolated by the plurality of reagents in the kit is about 500 μg to about 2 μg. In some embodiments, the amount of cellular DNA isolated by the plurality of reagents in the kit is about 200 ng to about 500 ng. In some embodiments, the region of the RAD9 gene comprises a transcription suppressor domain of intron 2. In some embodiments, the kit further comprises instructions indicating a threshold level of methylation above which a diagnosis of cancer can be made in the subject, wherein the threshold is at least about 1.5 times greater or about 1.5 to about 4.5 times greater methylation of the plurality of CpG sites within the region of the RAD9 gene in DNA present in the urine sample from the subject compared to an amount of methylation at a plurality of the same CpG sites in the same region of the RAD9 gene in DNA present in a second urine sample obtained from a healthy subject that does not have cancer or in a cohort of urine samples obtained from a plurality of healthy subjects that do not have cancer.

In another aspect, described herein is a method of treating prostate, bladder, or kidney cancer in a subject comprising: (a) measuring methylation status at a plurality of CpG sites within a region of a RAD9 gene in DNA present in a urine sample from the subject; and (b) administering an effective amount of a prostate, bladder, or kidney cancer treatment to the subject who has been determined to have an increased level of methylation at the plurality of CpG sites, thereby treating prostate, bladder, or kidney cancer in the subject.

In some embodiments, the subject is a human. In some embodiments, measuring the amount of the methylation comprises performing bisulfite sequencing or bisulfite pyrosequencing. In some embodiments, the methylation status of the plurality of CpG sites within a region of a RAD9 gene is determined in cell-free DNA, cellular DNA, or total DNA present in the urine sample. In some embodiments, DNA present in the urine sample from the subject is isolated prior to measuring the methylation status of the plurality of CpG sites. In some embodiments, the isolated DNA comprises an amount of cell-free DNA from the urine sample. In some embodiments, the amount of cell-free DNA isolated from the urine sample is about 500 μg to about 2 μg. In some embodiments, the amount of cell-free DNA isolated from the urine sample is about 200 ng to about 500 ng. In some embodiments, the isolated DNA comprises an amount of cellular DNA from the urine sample. In some embodiments, the amount of cellular DNA isolated from the urine sample is about 500 μg to about 2 μg. In some embodiments, the amount of cellular DNA isolated from the urine sample is about 200 ng to about 500 ng. In some embodiments, the region of the RAD9 gene comprises a transcription suppressor domain of intron 2. In some embodiments, the plurality of CpG sites comprises sites 404 to 518 within intron 2. In some embodiments, the subject is determined to have an increased level of methylation at the plurality of CpG sites if the amount of methylation of the plurality of CpG sites within the region of the RAD9 gene in DNA present in a urine sample from the subject is at least about 1.5 times greater or about 1.5 to about 4.5 times greater methylation compared to an amount of methylation at a plurality of the same CpG sites in the same region of the RAD9 gene in DNA present in a second urine sample obtained from a healthy subject that does not have cancer or in a cohort of urine samples obtained from a plurality of healthy subjects that do not have cancer. In some embodiments, the prostate, bladder or kidney cancer treatment is surgery, transurethral resection of bladder tumor (TURBT), radiation therapy, Lutetium-PSMA, radium-223 (Xofigo), cryotherapy, high-intensity focused ultrasound (HIFU), focal laser ablation, chemotherapy, immunotherapy, hormonal therapy, orchiectomy, administration of antineoplastic agents, or a combination thereof. In some embodiments, the immunotherapy comprises administering a checkpoint inhibitor, a bi-specific antibody, an antibody-drug conjugate, a cytokine, or performing an intravesical immunotherapy. In some embodiments, the cytokine is IL-2. In some embodiments, the intravesical immunotherapy is Bacillus Calmette-Guerin (BSG). In some embodiments, the administration of antineoplastic agents comprises administering a PARP inhibitor.

In another aspect, described herein is a method for diagnosing cancer in a subject, the method comprising: determining a methylation status at a plurality of CpG sites within a region of a RAD9 gene in DNA present in a urine sample from the subject, wherein an amount of methylation of the plurality of CpG sites that exceeds a threshold indicates that the subject has cancer.

In some embodiments, the subject is a human. In some embodiments, determining the amount of the methylation comprises performing bisulfite sequencing, bisulfite pyrosequencing, methylation specific restriction endonucleases (MSRE) analysis, methylation specific high-resolution DNA melting (MS-HRM), or quantitative methylation specific polymerase chain reaction (qMSP). In some embodiments, determining the amount of the methylation comprises performing bisulfite pyrosequencing. In some embodiments, the methylation status of the plurality of CpG sites within the region of the RAD9 gene is determined in cell-free DNA, cellular DNA, or total DNA present in the urine sample. In some embodiments, DNA present in the urine sample from the subject is isolated prior to determining the methylation status of the plurality of CpG sites. In some embodiments, the isolated DNA comprises an amount of cell-free DNA from the urine sample. In some embodiments, the amount of cell-free DNA isolated from the urine sample is about 500 μg to about 2 μg. In some embodiments, the amount of cell-free DNA isolated from the urine sample is about 200 ng to about 500 ng. In some embodiments, the isolated DNA comprises an amount of cellular DNA from the urine sample. In some embodiments, the amount of cellular DNA isolated from the urine sample is about 500 μg to about 2 μg. In some embodiments, the amount of cellular DNA isolated from the urine sample is about 200 ng to about 500 ng. In some embodiments, the RAD9 gene comprises a transcription suppressor domain of intron 2. In some embodiments, the plurality of CpG sites comprises sites 404 to 518 within intron 2. In some embodiments, the cancer is prostate, bladder, or kidney cancer. In some embodiments, the method further comprises administering a cancer treatment to the subject. In some embodiments, the threshold is at least about 1.5 times greater or about 1.5 to about 4.5 times greater methylation of the plurality of CpG sites within the region of the RAD9 gene in DNA present in the urine sample from the subject, compared to an amount of methylation at a plurality of the same CpG sites in the same region of the RAD9 gene in DNA present in a second urine sample obtained from a healthy subject that does not have cancer or in a cohort of urine samples obtained from a plurality of healthy subjects that do not have cancer.

In another aspect, described herein is a nucleic acid primer comprising SEQ ID NO: 2.

In another aspect, described herein is a nucleic acid primer comprising SEQ ID NO: 3, wherein the nucleic acid primer is biotinylated.

In another aspect, described herein is a composition comprising a nucleic acid primer comprising SEQ ID NO: 2.

In some embodiments, the composition further comprises a biotinylated nucleic acid primer comprising SEQ ID NO: 3.

In another aspect, described herein is a composition comprising a biotinylated nucleic acid primer comprising SEQ ID NO: 3.

In another aspect, described herein is a composition comprising a complex, wherein the complex comprises a modified RAD9 DNA molecule, wherein unmethylated cytosine residues present in DNA present in a urine sample from a subject have been modified; and a primer hybridized to the modified RAD9 DNA molecule.

In some embodiments, the composition further comprises a recombinant thermostable DNA polymerase bound to the complex. In some embodiments, the primer further comprises one or more biotin molecules covalently attached to the primer. In some embodiments, the modified RAD9 DNA molecule has been amplified by PCR. In some embodiments, the primer comprises SEQ ID NO:2 or 3. In some embodiments, the primer comprises SEQ ID NO: 4 or 5. In some embodiments, the modified RAD9 DNA molecule comprises the transcription suppressor domain of intron 2. In some embodiments, the complex further comprises a second primer hybridized to the modified RAD9 DNA molecule. In some embodiments, the primer comprises SEQ ID NO: 2 and the second primer comprises SEQ ID NO: 3, or wherein the primer comprises SEQ ID NO: 3 and the second primer comprises SEQ ID NO: 2. In some embodiments, the primer comprises SEQ ID NO: 4 and the second primer comprises SEQ ID NO: 5, or wherein the primer comprises SEQ ID NO: 5 and the second primer comprises SEQ ID NO: 4.

### BRIEF DESCRIPTION OF THE DRAWINGS

The following figures depict illustrative embodiments of the invention.

FIG. 1 is a diagram showing the RAD9 regulatory network.

FIG. 2 is a diagram showing DNMT1 and DNMT3B methylate cytosines in CpG sites within a RAD9 transcription suppressor region to activate transcription.

FIGS. 3A-3B are chemical structures of cytosine (FIG. 3A) and 5-methylcytosine (FIG. 3B).

FIG. 4 is a flow chart showing the process of bisulfite sequencing.

FIG. 5 is a graph showing methylation of cytosines at RAD9 transcription suppressor CpG sites using bisulfite sequencing.

FIG. 6 is a diagram showing a bisulfite pyrosequencing strategy for detection of methylated cytosines in RAD9 CpG sites.

FIGS. 7A-7B are graphs showing RAD9 CpG site methylation in normal human prostate tissues (FIG. 7A) and in human prostate tumors (FIG. 7B) using bisulfite sequencing.

FIG. 8 is a flow chart showing the process of bisulfite pyrosequencing.

FIG. 9 is a graph showing the percent of 9 CpG sites located within the transcription suppressor region of the RAD9 gene from the cell-free fraction of human urine for two healthy controls (Healthy Control 1 and Healthy Control 2) and two prostate cancer patients (Patient 3 and Patient 4a/4b).

### DETAILED DESCRIPTION

All patent applications, published patent applications, issued and granted patents, texts, and literature references cited in this specification are hereby incorporated herein by reference in their entirety to more fully describe the state of the art to which the present disclosed subject matter pertains.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Described herein are diagnostic and prognostic biomarkers for men with prostate cancer. Specific methylation status at a plurality of CpG sites within a region of a RAD9 gene in DNA have been identified as markers for certain forms of cancer.

The term “subject” is used throughout the specification to describe an animal, human or non-human, to whom treatment according to the methods of the present invention is provided. A subject may be, for example, a mammal such as a dog, a cat, a cow, a horse, a rabbit, a monkey, a pig, a sheep, a goat, a mouse, a rat, or a human.

The singular forms “a,” “an,” and “the” include plural reference unless the context clearly dictates otherwise.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein “RAD9” refers to the gene that encodes the cell cycle checkpoint control protein RAD9.

As used herein “DNMT1” refers to DNA-methyltransferase.

As used herein “DNMT3B” refers to DNA-methyltransferase 3 beta.

As used herein “CpG sites” refers to regions of DNA where a cytosine nucleotide is followed by a guanine nucleotide in the linear sequence of bases along its 5′→3′ direction. ^{m}CpG and CpG refer to methylated and unmethylated CpG sites respectively.

In some embodiments, the disclosure is directed to an assay that detects RAD9 gene methylation in urine and is a non-invasive diagnostic and prognostic test for prostate cancer. In some embodiments, the assay measures the levels of RAD9 methylation (detection and/or quantification) from tumor DNA in urine. In some embodiments, the assay provides a non-invasive method with greater sensitivity for the detection of prostate cancer, particularly for aggressive variants, and may complement other diagnostic methods such as the serum PSA assay. Aberrant overexpression and hypermethylation of RAD9 has been shown to drive prostate cancer, and a non-invasive test based on RAD9 could readily complement the PSA assay to improve the rate of detection for prostate cancer. In some embodiments, the assays described herein may also be used to diagnose bladder, kidney, or other cancers in a human subject.

In some embodiments, described herein is a point-of-care non-invasive prognostic test for prostate cancer. In some embodiments, the non-invasive methods, assays, devices, kits, nucleic acids and compositions of the invention can be used for prostate cancer grading. In some embodiments, the methods, assays, devices, kits, nucleic acids and compositions described herein can be used as a research tool for the identification of prostate cancer biomarkers. In some embodiments, the methods, assays, devices, kits, nucleic acids, and compositions described herein can be used as a research tool for the identification of bladder or kidney cancer biomarkers. In some embodiments, diagnosis is confirmed following assay detection by additional pathological analysis of prostate tissue obtained from a biopsy.

In some embodiments, the novel methods, assays, devices, kits, nucleic acids and compositions described herein offer non-invasive, highly accessible point-of-care tests that limit reliance on expensive advanced imaging studies (e.g., Axumin, PSMA-PET, nuclear bone scans) to monitor response to therapy and progression of disease. In some embodiments, the disclosure is directed to an assay that uses RAD9 intron 2 hypermethylation as a urine prostate cancer biomarker. An advantage of measuring DNA in urine is that DNA is very stable compared to RNA or protein abundance and quantities of DNA can be amplified. Additionally, urine is easily acquired non-invasively, and thus is an ideal source for “liquid biopsy.” Another advantage is that urine contains exfoliated tumor cells and cell-free tumor DNA that can be leveraged in the non-invasive diagnostic and/or prognostic assays described herein.

Described herein are novel, non-invasive diagnostic and prognostic biomarker-based methods, assays, devices, kits, nucleic acids and compositions for men with prostate cancer. In some embodiments, assays, devices, kits, nucleic acids and compositions are used for detection of metastatic castrate-resistant prostate cancer (CRPC) and/or aggressive-variant histologies where PSA and/or other histology-specific biomarkers (e.g., chromogranin A for neuroendocrine prostate cancer) are of limited value. Urine contains prostate cancer tumor cells and cell-free tumor DNA. In some embodiments, the present disclosure leverages urine as a “liquid biopsy” for prostate cancer using a RAD9 methylation assay for urine specimens from patients with high volume metastatic CRPC and all stages of aggressive-variant histologies, including neuroendocrine subtypes, compared to age-matched, cancer-free controls.

### RAD9 in Prostate Cancer

When cells are exposed to physical or chemical agents that damage DNA, or develop damage during normal metabolic processes, deleterious effects can ensue, including mutation, cancer, or death. However, cellular mechanisms exist to repair the damage, stabilize the genome, and neutralize harmful effects. RAD9 plays a prominent role in processes that promote survival and genomic integrity. RAD9 is a DNA damage response protein critical for prostate carcinogenesis. Aberrant overexpression of the gene and dysregulated by methylation can drive prostate cancer. There is a highly significant direct association between stage of cancer and frequency/intensity of RAD9 immunohistochemical staining as well as RAD9 gene hypermethylation in human prostate biopsy. For example, human prostate cancer cell lines (e.g., CWR22, DU145, LNCaP, PC-3) have very high levels of RAD9 protein relative to non-cancer prostate cells (PrEC) (e.g., 7.8-fold to 15.5-fold higher). The knockdown or knockout of RAD9 in human prostate cancer cells reduces or eliminates the ability of cells to form tumors in mouse xenographs, reduces cancer-related in vitro cellular phenotypes such as rapid cell migration, anchorage-independent growth, and anoikis resistance, and alters cancer related molecular activities such as integrin 31 level, AKT activation, and AGR2 expression. Without intending to be bound by any particular theory, these data suggest that the abundance of RAD9, and not a mutation in the RAD9 gene, is critical to support neoplastic processes.

This evolutionarily conserved gene (RAD9) has multiple functions needed for the cellular response to DNA damage, including cell cycle checkpoints, DNA repair, and pro-apoptotic activities. In addition, RAD9 can, like p53, act as a sequence specific transcription factor. RAD9 can bind p53 consensus sequences in the p21 promoter and cause transcription when overexpressed. There is strong evidence that an optimum level of RAD9 is needed for proper cell functioning, and too much or too little can be detrimental. For example, too much RAD9 protein can cause apoptosis or lead to prostate cancer. Too little RAD9 protein can cause cellular sensitivity to DNA damage, defects in DNA repair, and cell cycle checkpoints, and leads to the development of cataracts or skin cancer.

### DNMT1 and DNMT3B

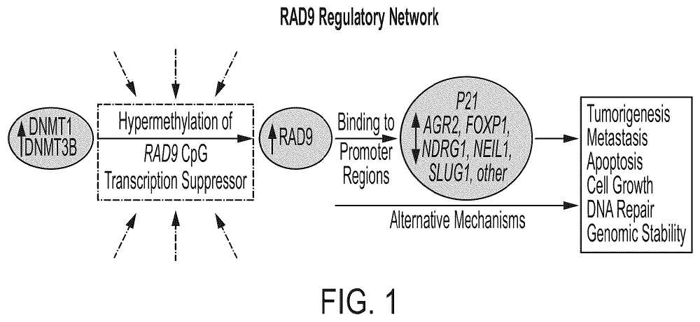

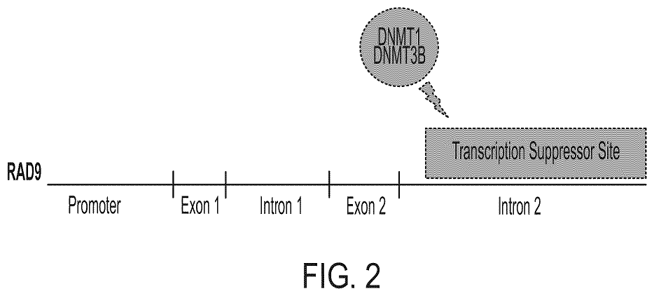

As shown in FIGS. 1-2, high expression of DNA methyltransferases DNMT1 and DNMT3B in prostate cancer cells leads to increased expression and hypermethylation of the RAD9 gene. Specifically, RAD9 is regulated epigenetically by DNMT1 and DNMT3B, via targeted hypermethylation, and consequent RAD9 overproduction promotes prostate tumorigenesis. Zhu, Aiping, et al. “DNMT1 and DNMT3B regulate tumorigenicity of human prostate cancer cells by controlling RAD9 expression through targeted methylation.” Carcinogenesis 42.2 (2021): 220-231, the content of which is hereby incorporated by reference in its entirety. DNA methyltransferases DNMT1 and DNMT3B levels are aberrantly high in several prostate cancer cell lines, and can methylate a “suppressor of transcription” region within intron 2 of the RAD9 gene in DU145, AVLA31 and AVLA41 cells de-repressing transcription and upregulating genes and subsequent transactivation or transrepression of downstream targets such as COX-2, FLT-1 P21, AGR2, FOXP1, NDRG1, NEIL1, SLUG1, and others (FIGS. 1-2). This can lead to various consequences in some circumstances including tumorigenesis, metastasis, apoptosis, cell growth, DNA repair, and/or genomic stability (FIG. 1). Without intending to be bound by any particular theory, there are three hypotheses: 1) DNMT1 and DNMT3B are highly expressed in certain prostate cancer cell lines because of chromatin structure, promoter methylation abnormalities, or mutation, and bind more tightly to RAD9 only in DU145, AVLA31 and AVLA41 cancer cell lines due to altered RAD9 chromatin; 2) RAD9 upregulation induces transcription of multiple downstream genes and pathways critical for genomic integrity, growth control, prostate carcinogenesis and metastasis; and 3) knock down of RAD9 expression reduces the metastastatic phenotype, including behaviors like migration towards fibronectin, invasion, cell adhesion to various extracellular matrix proteins, and metastatic behavior in an in vivo model.

### RAD9

RAD9 as described herein (also known as RAD9A or hRAD9) is located on Homo sapiens chromosome 11 (11q13.1) and has the GeneBank Gene ID: 5883. NCBI Reference Sequence: NC_000011.10 corresponds to the genomic sequence of RAD9, which is hereby incorporated by reference in its entirety. NCBI Reference Sequence: NM_004584.3 corresponds to the nucleotide sequence of transcript variant 1 of RAD9, which is hereby incorporated by reference in its entirety. Transcript variant 1 is 2086 base pairs in length and encodes NCBI Reference Sequence: NP_004575.1, which corresponds to the 391 amino acid polypeptide sequence of transcript variant 1 of RAD9, which is hereby incorporated by reference in its entirety. NCBI Reference Sequence: NM_001243224.1 corresponds to the nucleotide sequence of transcript variant 2 of RAD9 nucleotide sequence of transcript variant 1 of RAD9. Transcript variant 2 is 1992 base pairs in length and encodes NCBI Reference Sequence: NP_001230153.1, which corresponds to the 315 amino acid polypeptide sequence of transcript variant 2 of RAD9, which is hereby incorporated by reference in its entirety. Transcript variant 2 contains an alternate 5′ terminal exon (with an in-frame AUG) compared to variant 1. This results in a shorter isoform with a different N-terminus compared to transcript variant 1. The nucleotide and amino acid sequences can be readily obtained by one of ordinary skill in the art using the listed accession numbers.

The transcription suppressor domain (see FIG. 2) is within RAD9 intron 2 and is located at nucleic acid positions 67392418-67392608 (based on position within chromosome 11, GRCh38.p13 Primary Assembly) corresponding to nucleic acid positions 443-633 (based on position within RAD9, GeneBank Gene ID: 5883). The nucleic acid sequence of the transcription suppressor region of RAD9 is

(SEQ ID NO: 1)
CCCAGGGGGTGACGTGCACTTAGAGAAACTCGGGGAAGGCCTGGGTGTG
CGACCCCTCCTCTGCGGCAGCAGCGCCGGGGCCGACTCTGAAGGCTTCC
ATGGGGAAAGGAGGGTTTTTCAGCAGGTGGTGGCGGAGCGGGAGGACGA
TAGGGCAAGTGTGTGAGCAGAAGCAGCCAGAGGGCTGGGTCTGT.

Reference to RAD9 detailed above should be understood as a reference to all forms of RAD9 and to fragments or variants thereof. A person of skill in the art understands that some genes are known to exhibit allelic variation between individuals or single nucleotide polymorphisms. SNPs encompass insertions and deletions of varying size and simple sequence repeats, such as dinucleotide and trinucleotide repeats. Variants include nucleic acid sequences from the same region sharing at least 90%, 95%, 98%, 99% sequence identity i.e., having one or more deletions, additions, substitutions, inverted sequences etc. relative to the DNA regions described herein. Accordingly, the present invention should be understood to extend to such variants which, in terms of the present applications, achieve the same outcome despite the fact that minor genetic variations between the actual nucleic acid sequences may exist between individuals. The present invention should therefore be understood to extend to all forms of DNA which arise from any other mutation, polymorphic or allelic variation.

In terms of methods disclosed herein for assessing or detecting the methylation of one or more regions of RAD9, the methods can be designed to screen either the “plus” strand of the gene or the complementary “minus” strand. It is within the skill of the person in the art to choose which strand to analyze and to target that strand based on the chromosomal coordinates provided herein. In some embodiments, assays may be established to screen both strands.

### CpG Sites

As described herein, there are 9 CpG sites of interest within the transcription suppressor domain of RAD9 intron 2. SEQ ID NO: 1 comprises the RAD9 intron-containing DNA fragment with 9 target CpG sites. Table 1 shows correspondence of nucleic acid position for each CpG site based on three different numbering conventions: 1) nucleic acid position based on numbering in which position 1 is the “A” of the start codon ATG in RAD 9 (See Zhu, A., Zhang, X., and Lieberman, H. B. (2008) Rad9 has a functional role in human prostate carcinogenesis. Cancer Res. 68: 1267-1274, incorporated by reference in its entirety herein), 2) based on the GenBank ID: 5883 for RAD9, and 3) based the positions within chromosome 11 (GRCh38.p13 Primary Assembly)

TABLE 1
Nucleic acid positions for 9 CpG methylation
sites within RAD9 intron 2 suppressor domain

| | | 3. Chromosome 11, |
|---|---|---|
| | | GRCh38.p13 Primary |
| 1. RAD9 (+1 ATG) | 2. Gene Id: 5883 | Assembly |
| 404 | 473 | 67392448 |
| 423 | 492 | 67392467 |
| 437 | 506 | 67392481 |
| 446 | 515 | 67392490 |
| 449 | 518 | 67392493 |
| 455 | 524 | 67392499 |
| 505 | 574 | 67392549 |
| 510 | 579 | 67392554 |
| 518 | 587 | 67392562 |

Methylation status refers to the presence, absence and/or quantity of methylation at a particular nucleotide, or nucleotides, within a DNA region. The methylation status of a particular RAD9 sequence (e.g. RAD9 region as described herein) can indicate the methylation state of every base in the sequence or can indicate the methylation state of a subset of the base pairs (e.g., of the 9 cytosines of the CpG sites in Table 1) within the sequence, or can indicate information regarding regional methylation density within the sequence without providing precise information of where in the sequence the methylation occurs. The methylation status can be represented or indicated by a methylation value, which can be generated, for example, by quantifying the amount of cytosines of CpG sites in Table 1 that are methylated and can be used as a quantitative indicator of the methylation status. This is of useful when comparing the methylation status of a sequence in a sample to a threshold value.

In some embodiments, hypermethylation of the RAD9 gene has a strong positive correlation with prostate cancer stage. In some embodiments, a threshold detection difference in RAD9 (e.g., RAD9A) methylation between cancer versus cancer-free subject specimens is below about 0.5-fold, between about 0.5-fold and 1.5-fold, at least about 1.5-fold, between about 1.5-fold and about 10-fold, between about 1.5-fold and 4.5-fold, or between about 1.6-fold and 4.3-fold. In some embodiments, the threshold detection difference in RAD9 methylation between cancer and cancer-free subject specimens corresponds to between about 30% and 70% methylation in a target region of RAD9 in cancer subject specimen compared to between about 15% and 25% methylation in a target region of RAD9 in cancer-free subject specimen. In some embodiments, the threshold detection for RAD9 methylation in cancer is at least about 30% methylation. In some embodiments, the threshold detection for RAD9 methylation in cancer is at least about 30% to about 70% methylation. In some embodiments, the methylation level is the methylation level for the 9 CpG sites in Table 1.

Without being bound by theory, 9 CpG sites in Table 1 provide an improved diagnostic outcome relative to prior art methods and enable a screening method which can provide a high level of diagnostic specificity and/or a high level of diagnostic sensitivity. Sensitivity generally refers to the proportion of positive results which are correctly identified (i.e., the percentage of individuals correctly identified as having the disease in issue). Specificity generally refers to the proportion of negative results which are correctly identified (i.e., the percentage of individuals correctly identified as not having the disease in issue). Described herein are methods that exhibit a high level of specificity and/or a high level of sensitivity.

The methods described herein may refer to the comparison of the level of methylation of one or more RAD9 regions of a sample with the control methylation levels. The control level corresponds to the level of methylation of the one or more RAD9 regions of a corresponding sample that is not cancerous or in another biological sample from which DNA may be isolated for assay. In some embodiments, the control methylation level may be determined using non-cancerous tissues derived from the same individual who is the subject of the methods described herein. In some embodiments, the control methylation reflects the methylation level from an individual or collective results obtained from individuals other than the subject of the methods described herein. The control level may be calculated by any suitable means which would be well known to the person of skill in the art. For example, a population of normal tissues can be assessed in terms of the level of methylation of one or more RAD9 regions, thereby providing a standard value or range of values against which all future test samples are analyzed. In some embodiments, the control level may be determined from the subjects of a specific cohort and for use with respect to test samples derived from that cohort. Accordingly, there may be determined a number of standard values or ranges which correspond to cohorts which differ in respect of characteristics such as age, gender, ethnicity or health status. In some embodiments the control level may be a discrete level or a range of levels. An increase in the methylation level of one or more RAD9 regions in the subject relative to control levels is indicative of the tissue being cancerous. In some embodiments, urine RAD9 methylation is examined in three cohorts: 1) men with advanced CRPC; 2) men with NEPC; and 3) age-matched, cancer-free men. In some embodiments, the assays disclosed herein are used to detect disease progression in men with metastatic CRPC and aggressive-variant prostate cancer.

### Methods of Assessing Methylation Status

In one aspect, described herein is a method of assessing a methylation status of DNA present in a urine sample obtained from a human subject comprising assessing the methylation status at a plurality of methylation sites within one or more regions of a RAD9 gene. In some embodiments, the assessing comprises performing a bisulfite conversion of the one or more regions of the RAD9 gene, amplifying the converted one or more RAD9 regions or the complement thereof with primers comprising a nucleic acid sequence that is complementary to the converted one or more RAD9 regions under selective hybridization conditions, and detecting the methylation status of one or more cytosines in the one or more RAD9 regions. In some embodiments, the one or more regions of the RAD9 gene comprises a RAD region comprising transcription suppressor domain of intron 2. In some embodiments, detecting the methylation status of the one or more cytosines comprises detecting a methylation status of one or more cytosine residues comprising CpG sites 404 to 518 within intron 2. In some embodiments, the primers comprise SEQ ID NO: 2 and SEQ ID NO: 3. In some embodiments, detecting the methylation status of the one or more cytosines comprises sequencing the amplified converted RAD9 region using primers comprising SEQ ID NO: 4 or SEQ ID NO: 5.

In another aspect, described herein is a method of detecting methylation or unmethylation of a RAD9 DNA molecule of a human subject, the method comprising: (a) reacting an isolated RAD9 DNA molecule from a urine sample of the human subject with a bisulfite salt thereby forming a reacted RAD9 DNA molecule; (b) contacting the reacted RAD9 DNA molecule with a probe or a primer complementary to a sequence at or within 100 nucleotides of a plurality of cytosine methylation sites; and (c) detecting a presence or an absence of uracil in the reacted RAD9 DNA molecule at the plurality of cytosine methylation sites, thereby detecting methylation or unmethylation of the RAD9 DNA molecule of the human subject. In some embodiments, the method comprises isolating the RAD9 DNA molecule from a urine sample of the human subject. In some embodiments, the plurality of cytosine methylation sites is within a region of the RAD9 gene comprising transcription suppressor domain of intron 2. In some embodiments, the plurality of cytosine methylation sites comprises CpG sites 404 to 518 within intron 2. In some embodiments, the method further comprises determining whether a methylation level of the plurality of cytosine methylation sites is at least about 1.5 times greater or about 1.5 to about 4.5 times greater methylation in DNA molecules isolated from the urine sample of the human subject compared to an amount of methylation at a plurality of the same cytosine methylation sites in isolated RAD9 DNA molecules present in a second urine sample obtained from a healthy subject that does not have cancer or in a cohort of urine samples obtained from a plurality of healthy subjects that do not have cancer, and if the methylation level of the plurality of cytosine methylation sites is at least about 1.5 times greater or about 1.5 to about 4.5 times greater, administering to the human subject a treatment to treat or prevent prostate, bladder, or kidney cancer, wherein the treatment comprises surgery, radiation therapy, chemotherapy, immunotherapy, or administering an active agent comprising antineoplastic properties. In some embodiments, the administering to the human subject comprises administering a treatment to treat or prevent prostate cancer. In some embodiments, the active agent comprising antineoplastic properties is a PARP inhibitor. In some embodiments, the methylation level is below about 0.5-fold, between about 0.5-fold and 1.5-fold, at least about 1.5-fold, between about 1.5-fold and about 10-fold, between about 1.5-fold and 4.5-fold, or between about 1.6-fold and 4.3-fold greater. In some embodiments, the methylation level is at least about 30% methylation in a target region of RAD9. In some embodiments, the methylation level of the plurality of cytosine methylation sites is between at least about 30% methylation and about 70% methylation in a target region of RAD9.

In some embodiments, the urine sample is obtained from a human subject at risk of developing prostate, bladder, or kidney cancer. In some embodiments, the urine sample is obtained from a human subject at risk of developing prostate cancer. In some embodiments, the urine sample is obtained from a human subject at risk of developing a recurrence of prostate, bladder, or kidney cancer. In some embodiments, the urine sample is obtained from a human subject at risk of developing a recurrence of prostate cancer. In some embodiments, the urine sample is obtained from a human subject with prostate, bladder, or kidney cancer. In some embodiments, the urine sample is obtained from a human subject with prostate cancer.

In some embodiments, the method further comprising administering an effective amount of a prostate, bladder, or kidney cancer treatment to the subject whose methylation status has been determined to be an increased level of methylation within one or more regions of the RAD9 gene in accordance with the methods of treatment disclosed herein. For example, in some embodiments, the methylation status is of one or more cytosines in the one or more RAD9 regions. In some embodiments, the methylation status is of a plurality of CpG sites in the one or more RAD9 regions. In some embodiments, the one or more regions of the RAD9 gene comprises a RAD region comprising transcription suppressor domain of intron 2. In some embodiments, the methylation status of the one or more cytosines comprises detecting a methylation status of one or more cytosine residues comprising CpG sites 404 to 518 within intron 2. In some embodiments, the method further comprising administering to the human subject a treatment to treat or prevent prostate, bladder, or kidney cancer, wherein the treatment comprises surgery, radiation therapy, chemotherapy, immunotherapy, or administering an active agent comprising antineoplastic properties. In some embodiments, the method further comprising administering to the human subject a treatment to treat or prevent prostate cancer.

The methods of the present invention are useful as a one-time test or as an on-going monitor of subjects in need thereof. In some embodiments, a sample from a subject can be taken prior to initiating definitive radiotherapy with or without hormonal therapy and along the course of treatment, the methods to assess RAD9 methylation can be used as a predictive biomarker for response to therapy at these different timepoints. Such an embodiment is advantageous for subjects on hormonal therapy whose PSA is suppressed by the therapy rendering it uninterpretable from a predictive or prognostic sense. Without being bound by theory, RAD9 methylation may be uncoupled from androgen signaling, and thus is useful as a predictive biomarker for these subjects. In some embodiments, the method of assessing a methylation status of DNA present in a urine sample obtained from a human subject comprises monitoring a response to a therapy and/or progression of disease by assessing the methylation status at a plurality of methylation sites within one or more regions of a RAD9 gene at two or more time points. In some embodiments, the method comprises assessing the methylation status at a plurality of methylation sites within one or more regions of a RAD9 gene at a first point in time prior to administering a prostate, bladder, or kidney cancer treatment and at a second time point subsequent to said administering. In some embodiments, the method further comprises assessing the methylation status at a plurality of methylation sites within one or more regions of a RAD9 gene at one or more additional time points subsequent to said administering. In some embodiments, a reduction in the level of methylation within one or more regions of the RAD9 gene between the first time point and/or the second time point or one or more subsequent time points, if tested, indicates that the prostate, bladder, or kidney cancer treatment is effective. In some embodiments, a reduction in the level of methylation within one or more regions of the RAD9 gene between the second time point and one or more subsequent time points, if tested, indicates that the prostate, bladder, or kidney cancer treatment is effective. In some embodiments, no reduction or an increase in the level of methylation within one or more regions of the RAD9 gene between the first time point and/or the second time point or one or more subsequent time points, if tested, indicates that the prostate, bladder, or kidney cancer treatment is ineffective. In some embodiments, no reduction or an increase in the level of methylation within one or more regions of the RAD9 gene between the second time point and one or more subsequent time points, if tested, indicates that the prostate, bladder, or kidney cancer treatment is ineffective. In some embodiments, the assessing at any of the timepoints comprises performing a bisulfite conversion of the one or more regions of the RAD9 gene, amplifying the converted one or more RAD9 regions or the complement thereof with primers comprising a nucleic acid sequence that is complementary to the converted one or more RAD9 regions under selective hybridization conditions, and detecting the methylation status of one or more cytosines in the one or more RAD9 regions. In some embodiments, the one or more regions of the RAD9 gene comprises a RAD region comprising transcription suppressor domain of intron 2. In some embodiments, detecting the methylation status of the one or more cytosines comprises detecting a methylation status of one or more cytosine residues comprising CpG sites 404 to 518 within intron 2. In some embodiments, the primers comprise SEQ ID NO: 2 and SEQ ID NO: 3. In some embodiments, detecting the methylation status of the one or more cytosines comprises sequencing the amplified converted RAD9 region using primers comprising SEQ ID NO: 4 or SEQ ID NO: 5. In some embodiments, the prostate cancer treatment administered is definitive radiotherapy with hormonal therapy. In some embodiments, the prostate cancer treatment administered is definitive radiotherapy without hormonal therapy.

In some embodiments, the method comprises assessing the methylation status at a plurality of methylation sites within one or more regions of a RAD9 gene at a first point in time and at a second time point subsequent to said first point in time. In some embodiments, the method further comprises assessing the methylation status at a plurality of methylation sites within one or more regions of a RAD9 gene at one or more additional time points subsequent to said second point in time. In some embodiments, the urine sample is obtained from a human subject at risk of developing prostate, bladder, or kidney cancer. In some embodiments, the urine sample is obtained from a human subject at risk of developing prostate cancer. In some embodiments, the urine sample is obtained from a human subject at risk of developing a recurrence of prostate, bladder, or kidney cancer. In some embodiments, the urine sample is obtained from a human subject at risk of developing a recurrence of prostate cancer. In some embodiments, the urine sample is obtained from a human subject with prostate, bladder, or kidney cancer. In some embodiments, the urine sample is obtained from a human subject with prostate cancer. In some embodiments, maintenance or reduction in the level of methylation within one or more regions of the RAD9 gene between the first time point and/or the second time point or one or more subsequent time points, if tested, indicates that a prostate, bladder, or kidney cancer in the subject is not progressing or worsening. In some embodiments, maintenance or reduction in the level of methylation within one or more regions of the RAD9 gene between the first time point and/or the second time point or one or more subsequent time points, if tested, indicates that the risk of developing or having a recurrence of prostate, bladder, or kidney cancer in the subject is low. In some embodiments, maintenance or reduction in the level of methylation within one or more regions of the RAD9 gene between the second time point and one or more subsequent time points, if tested, indicates that a prostate, bladder, or kidney cancer in the subject is not progressing or worsening. In some embodiments, maintenance or reduction in the level of methylation within one or more regions of the RAD9 gene between the second time point and one or more subsequent time points, if tested, indicates that the risk of developing or having a recurrence of prostate, bladder, or kidney cancer in the subject is low. In some embodiments, an increase in the level of methylation within one or more regions of the RAD9 gene between the first time point and/or the second time point or one or more subsequent time points, if tested, indicates that a prostate, bladder, or kidney cancer in the subject is worsening. In some embodiments, an increase in the level of methylation within one or more regions of the RAD9 gene between the first time point and/or the second time point or one or more subsequent time points, if tested, indicates that the subject is at risk of developing or having a recurrence of prostate, bladder, or kidney cancer. In some embodiments, an increase in the level of methylation within one or more regions of the RAD9 gene between the second time point and one or more subsequent time points, if tested, indicates that a prostate, bladder, or kidney cancer in the subject is worsening. In some embodiments, an increase in the level of methylation within one or more regions of the RAD9 gene between the second time point and one or more subsequent time points, if tested, indicates that the subject is at risk of developing or having a recurrence of prostate, bladder, or kidney cancer. In some embodiments, the assessing at any of the timepoints comprises performing a bisulfite conversion of the one or more regions of the RAD9 gene, amplifying the converted one or more RAD9 regions or the complement thereof with primers comprising a nucleic acid sequence that is complementary to the converted one or more RAD9 regions under selective hybridization conditions, and detecting the methylation status of one or more cytosines in the one or more RAD9 regions. In some embodiments, the one or more regions of the RAD9 gene comprises a RAD region comprising transcription suppressor domain of intron 2. In some embodiments, detecting the methylation status of the one or more cytosines comprises detecting a methylation status of one or more cytosine residues comprising CpG sites 404 to 518 within intron 2. In some embodiments, the primers comprise SEQ ID NO: 2 and SEQ ID NO: 3. In some embodiments, detecting the methylation status of the one or more cytosines comprises sequencing the amplified converted RAD9 region using primers comprising SEQ ID NO: 4 or SEQ ID NO: 5.

In some embodiments, in the methods of assessing described here, the second time point is 1 week, 2 weeks, 3 weeks, 4 weeks, 5 weeks, 6 weeks, 7 weeks, 8 weeks, 9 weeks, 10 weeks, 11 weeks, 12 weeks, after first administering a prostate, bladder, or kidney cancer treatment. In some embodiments, in the methods of assessing described here, the second time point is 1 week, 2 weeks, 3 weeks, 4 weeks, 5 weeks, 6 weeks, 7 weeks, 8 weeks, 9 weeks, 10 weeks, 11 weeks, 12 weeks, 1 month, 2 months, 3 months, 4 months, 5 months, 6 months, 7 months, 8 months, 9 months, 10 months, 11 months, or 12 months after the first time point. In some embodiments, in the methods of assessing described here, the one or more subsequent time points are performed every 1 week, 2 weeks, 3 weeks, 4 weeks, 5 weeks, 6 weeks, 7 weeks, 8 weeks, 9 weeks, 10 weeks, 11 weeks, 12 weeks, 1 month, 2 months, 3 months, 4 months, 5 months, 6 months, 7 months, 8 months, 9 months, 10 months, 11 months, or 12 months after the second time point.

In certain aspects, the methods described herein for assessing a methylation status of DNA and/or for detecting methylation or unmethylation of a RAD9 DNA molecule of a human subject, can be performed on DNA obtained from a blood, plasma or serum sample of a human subject. In some embodiments, the method comprises isolating the RAD9 DNA molecule from a blood, plasma, serum, or urine sample of the human subject. The sample can be any biological sample derived from a subject, which contains nucleic acids or polypeptides. Non-limiting examples of samples include blood, plasma, serum, or urine. The sample can be collected according to conventional techniques and used directly for analysis or stored. The sample can be treated prior to performing the method, in order to render or improve availability of nucleic acids for testing. Treatments include, for instance, lysis (e.g., mechanical, physical, or chemical), centrifugation. The nucleic acids can be pre-purified or enriched by conventional techniques, and/or reduced in complexity. Nucleic acids can also be treated with enzymes or other chemical or physical treatments to produce fragments thereof. In one embodiment, the sample is contacted with reagents, such as probes, primers, or ligands, in order to assess the methylated RAD9 nucleic acid. Contacting can be performed in any suitable device, such as a plate, tube, well, or glass. In some embodiments, the contacting is performed on a substrate coated with the reagent, such as an antibody or epitope binding fragment of an antibody. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids of the sample.

### Methods of Treating

In another aspect, described herein is a method of treating prostate, bladder, or kidney cancer in a subject comprising: (a) measuring methylation status at a plurality of CpG sites within a region of a RAD9 gene in DNA present in a urine sample from the subject; and (b) administering an effective amount of a prostate, bladder, or kidney cancer treatment to the subject who has been determined to have an increased level of methylation at the plurality of CpG sites, thereby treating prostate, bladder, or kidney cancer in the subject. In some embodiments, the subject is a human. In some embodiments, measuring the amount of the methylation comprises performing bisulfite sequencing or bisulfite pyrosequencing. In some embodiments, the methylation status of the plurality of CpG sites within a region of a RAD9 gene is determined in cell-free DNA, cellular DNA, or total DNA present in the urine sample. In some embodiments, DNA present in the urine sample from the subject is isolated prior to measuring the methylation status of the plurality of CpG sites. In some embodiments, the isolated DNA comprises an amount of cell-free DNA from the urine sample. In some embodiments, the amount of cell-free DNA isolated from the urine sample is about 500 μg to about 2 μg. In some embodiments, the amount of cell-free DNA isolated from the urine sample is about 200 ng to about 500 ng. In some embodiments, the isolated DNA comprises an amount of cellular DNA from the urine sample. In some embodiments, the amount of cellular DNA isolated from the urine sample is about 500 μg to about 2 μg. In some embodiments, the amount of cellular DNA isolated from the urine sample is about 200 ng to about 500 ng. In some embodiments, the region of the RAD9 gene comprises a transcription suppressor domain of intron 2. In some embodiments, the plurality of CpG sites comprises sites 404 to 518 within intron 2. In some embodiments, the subject is determined to have an increased level of methylation at the plurality of CpG sites if the amount of methylation of the plurality of CpG sites within the region of the RAD9 gene in DNA present in a urine sample from the subject is at least about 1.5 times greater or about 1.5 to about 4.5 times greater methylation compared to an amount of methylation at a plurality of the same CpG sites in the same region of the RAD9 gene in DNA present in a second urine sample obtained from a healthy subject that does not have cancer or in a cohort of urine samples obtained from a plurality of healthy subjects that do not have cancer. In some embodiments, the prostate, bladder or kidney cancer treatment is surgery, transurethral resection of bladder tumor (TURBT), radiation therapy, Lutetium-PSMA, radium-223 (Xofigo), cryotherapy, high-intensity focused ultrasound (HIFU), focal laser ablation, chemotherapy, immunotherapy, hormonal therapy, orchiectomy, administration of antineoplastic agents, or a combination thereof. In some embodiments, the immunotherapy comprises administering a checkpoint inhibitor, a bi-specific antibody, an antibody-drug conjugate, a cytokine, or performing an intravesical immunotherapy. In some embodiments, the cytokine is IL-2. In some embodiments, the intravesical immunotherapy is Bacillus Calmette-Guerin (BSG). In some embodiments, the administration of antineoplastic agents comprises administering a PARP inhibitor. In some embodiments, the administration is an effective amount of a prostate cancer treatment. In some embodiments, the methylation level is below about 0.5-fold, between about 0.5-fold and 1.5-fold, at least about 1.5-fold, between about 1.5-fold and about 10-fold, between about 1.5-fold and 4.5-fold, or between about 1.6-fold and 4.3-fold greater. In some embodiments, the methylation level is at least about 30% methylation in a target region of RAD9. In some embodiments, the methylation level of the plurality of cytosine methylation sites is between at least about 30% methylation and about 70% methylation in a target region of RAD9.

IL-2 is an approved treatment for metastatic renal cell carcinoma and metastatic melanoma known in the art. See Jiang T, Zhou C, Ren S. Role of IL-2 in cancer immunotherapy. Oncoimmunology. 2016 Apr. 25; 5(6):e1163462. Bacillus Calmette-Guerin (BCG) is an approved first line therapy for patients with nonmuscle invasive bladder cancer known in the art. See Guallar-Garrido S, Julián E. Bacillus Calmette-Guérin (BCG) Therapy for Bladder Cancer: An Update. Immunotargets Ther. 2020 Feb. 13; 9:1-11. PARP inhibitors Olaparib and Rucaparib are approved for treating metastatic castration-resistant prostate cancer. See Congregado B, Rivero I, Osmán I, Sáez C, Medina López R. PARP Inhibitors: A New Horizon for Patients with Prostate Cancer. Biomedicines. 2022 Jun. 15; 10(6):1416. Additional PARP inhibitors are disclosed in Table 1 of Congregado B, Rivero I, Osmán I, Sáez C, Medina López R. PARP Inhibitors: A New Horizon for Patients with Prostate Cancer. Biomedicines. 2022 Jun. 15; 10(6):1416 incorporated herein by reference. The treatments disclosed herein can be used in combination with other treatments. See e.g., Tables 2-4 of Congregado B et al.

In some embodiments, the urine sample is obtained from a human subject at risk of developing prostate, bladder, or kidney cancer. In some embodiments, the urine sample is obtained from a human subject at risk of developing prostate cancer. In some embodiments, the urine sample is obtained from a human subject at risk of developing a recurrence of prostate, bladder, or kidney cancer. In some embodiments, the urine sample is obtained from a human subject at risk of developing a recurrence of prostate cancer. In some embodiments, the urine sample is obtained from a human subject with prostate, bladder, or kidney cancer. In some embodiments, the urine sample is obtained from a human subject with prostate cancer.

In some embodiments, measuring methylation status at a plurality of CpG sites within a region of a RAD9 gene in DNA present in a urine, blood, plasma, or serum sample from the subject of the method is performed in accordance with the methods of assessing methylation status described herein.

The treatments described herein can be administered to the subject once. Alternatively, treatments can be administered once or twice daily to a subject in need thereof for a period of from about two to about twenty-eight days, or from about seven to about ten days. A treatment can also be administered once or twice daily to a subject for a period of 1, 2, 3, 4, 5, 6, 7, 8, 9, 10, 11, 12 times per year, or a combination thereof. Furthermore, therapeutics can be co-administrated with another therapeutic.

The treatments of this invention can be formulated and administered to reduce the symptoms associated with prostate, bladder, or kidney cancer. Treatments can be administered by any conventional means available for use in conjunction with pharmaceuticals. Treatments can be administered alone, but are generally administered with a pharmaceutical carrier selected on the basis of the chosen route of administration and standard pharmaceutical practice.

A therapeutically effective treatment can depend upon a number of factors known to those or ordinary skill in the art. The dose(s) of a treatment inhibitor can vary, for example, depending upon the identity, size, and condition of the subject or sample being treated, further depending upon the route by which the treatment is to be administered. These amounts can be readily determined by a skilled artisan. Any of the therapeutic applications described herein can be applied to any subject in need of such therapy, including, for example, a mammal such as a dog, a cat, a cow, a horse, a rabbit, a monkey, a pig, a sheep, a goat, or a human.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa (20th Ed., 2000), the entire disclosure of which is herein incorporated by reference.

### Methods of Diagnosis

In another aspect, described herein is a method for diagnosing cancer in a subject, the method comprising: determining a methylation status at a plurality of CpG sites within a region of a RAD9 gene in DNA present in a blood, plasma, serum, or urine sample from the subject, wherein an amount of methylation of the plurality of CpG sites that exceeds a threshold indicates that the subject has cancer. In some embodiments, the subject is a human. In some embodiments, determining the amount of the methylation comprises performing bisulfite sequencing, bisulfite pyrosequencing, methylation specific restriction endonucleases (MSRE) analysis, methylation specific high-resolution DNA melting (MS-HRM), or quantitative methylation specific polymerase chain reaction (qMSP). In some embodiments, determining the amount of the methylation comprises performing bisulfite pyrosequencing. In some embodiments, the methylation status of the plurality of CpG sites within the region of the RAD9 gene is determined in cell-free DNA, cellular DNA, or total DNA present in the urine sample. In some embodiments, DNA present in the urine sample from the subject is isolated prior to determining the methylation status of the plurality of CpG sites. In some embodiments, the isolated DNA comprises an amount of cell-free DNA from the urine sample. In some embodiments, the amount of cell-free DNA isolated from the urine sample is about 500 μg to about 2 kg. In some embodiments, the amount of cell-free DNA isolated from the urine sample is about 200 ng to about 500 ng. In some embodiments, the isolated DNA comprises an amount of cellular DNA from the urine sample. In some embodiments, the amount of cellular DNA isolated from the urine sample is about 500 μg to about 2 kg. In some embodiments, the amount of cellular DNA isolated from the urine sample is about 200 ng to about 500 ng. In some embodiments, the RAD9 gene comprises a transcription suppressor domain of intron 2. In some embodiments, the plurality of CpG sites comprises sites 404 to 518 within intron 2. In some embodiments, the cancer is prostate, bladder, or kidney cancer. In some embodiments, the method further comprises administering a cancer treatment to the subject. In some embodiments, the threshold is at least about 1.5 times greater or about 1.5 to about 4.5 times greater methylation of the plurality of CpG sites within the region of the RAD9 gene in DNA present in the urine sample from the subject, compared to an amount of methylation at a plurality of the same CpG sites in the same region of the RAD9 gene in DNA present in a second urine sample obtained from a healthy subject that does not have cancer or in a cohort of urine samples obtained from a plurality of healthy subjects that do not have cancer. In some embodiments, the threshold is below about 0.5-fold, between about 0.5-fold and 1.5-fold, at least about 1.5-fold, between about 1.5-fold and about 10-fold, between about 1.5-fold and 4.5-fold, or between about 1.6-fold and 4.3-fold greater. In some embodiments, the threshold is at least about 30% methylation in a target region of RAD9. In some embodiments, the threshold is between at least about 30% methylation and about 70% methylation in a target region of RAD9.

In certain aspects, the methods described herein for diagnosing cancer in a subject can be performed on DNA obtained from a blood, plasma or serum sample of a human subject. In some embodiments, the method comprises isolating the RAD9 DNA molecule from a blood, plasma, serum, or urine sample of the human subject. The sample can be any biological sample derived from a subject, which contains nucleic acids or polypeptides. Non-limiting examples of samples include blood, plasma, serum, or urine. The sample can be collected according to conventional techniques and used directly for analysis or stored. The sample can be treated prior to performing the method, in order to render or improve availability of nucleic acids for testing. Treatments include, for instance, lysis (e.g., mechanical, physical, or chemical), centrifugation. The nucleic acids can be pre-purified or enriched by conventional techniques, and/or reduced in complexity. Nucleic acids can also be treated with enzymes or other chemical or physical treatments to produce fragments thereof. In one embodiment, the sample is contacted with reagents, such as probes, primers, or ligands, in order to assess the methylated RAD9 nucleic acid. Contacting can be performed in any suitable device, such as a plate, tube, well, or glass. In some embodiments, the contacting is performed on a substrate coated with the reagent, such as an antibody or epitope binding fragment of an antibody. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids of the sample.

### Kits, Nucleic Acids, and Compositions

In another aspect, described herein is a kit for detecting a cytosine methylation of a region of a RAD9 gene, the kit comprising: a primer set comprising a plurality of forward and reverse primers designed to amplify one or more partially methylated forms of a DNA form of the RAD9 gene in which unmethylated cytosine residues present in DNA present in a blood, plasma, serum, or urine sample from a subject have been modified, wherein the primer set includes primers comprising SEQ ID NO: 2 and SEQ ID NO: 3. In some embodiments, the nucleic acid primer comprises a sequence is about 70%, about 75%, about 80%, about 85%, about 90%, about 93%, about 95%, about 97%, about 98%, or about 99% identical to SEQ ID NO: 2. In some embodiments, the nucleic acid primer comprises a sequence is about 70%, about 75%, about 80%, about 85%, about 90%, about 93%, about 95%, about 97%, about 98%, or about 99% identical to SEQ ID NO: 3. In some embodiments, the kit additionally comprises an agent which modifies unmethylated cytosine residues. In some embodiments, the agent is a bisulfite salt. In some embodiments, the kit additionally comprises a plurality of reagents configured to effect DNA amplification and/or detection. In some embodiments, the primer set is directed to detecting methylation at a plurality of CpG sites comprising 404 to 518 within intron 2 of RAD9. In some embodiments, the subject is a human. In some embodiments, the kit additionally comprises a plurality of reagents configured to perform bisulfite sequencing or bisulfite pyrosequencing. In some embodiments, the kit additionally comprises a plurality of reagents configured to isolate an amount of cell-free DNA from the urine sample. In some embodiments, the amount of cell-free DNA isolated by the plurality of reagents in the kit is about 500 μg to about 2 μg. In some embodiments, the amount of cell-free DNA isolated by the plurality of reagents in the kit is about 200 ng to about 500 ng. In some embodiments, the kit additionally comprises a plurality of reagents configured to isolate an amount of cellular DNA from the urine sample. In some embodiments, the amount of cellular DNA isolated by the plurality of reagents in the kit is about 500 μg to about 2 μg. In some embodiments, the amount of cellular DNA isolated by the plurality of reagents in the kit is about 200 ng to about 500 ng. In some embodiments, the region of the RAD9 gene comprises a transcription suppressor domain of intron 2. In some embodiments, the kit further comprises instructions indicating a threshold level of methylation above which a diagnosis of cancer can be made in the subject, wherein the threshold is at least about 1.5 times greater or about 1.5 to about 4.5 times greater methylation of the plurality of CpG sites within the region of the RAD9 gene in DNA present in the urine sample from the subject compared to an amount of methylation at a plurality of the same CpG sites in the same region of the RAD9 gene in DNA present in a second urine sample obtained from a healthy subject that does not have cancer or in a cohort of urine samples obtained from a plurality of healthy subjects that do not have cancer. In some embodiments, the threshold is below about 0.5-fold, between about 0.5-fold and 1.5-fold, at least about 1.5-fold, between about 1.5-fold and about 10-fold, between about 1.5-fold and 4.5-fold, or between about 1.6-fold and 4.3-fold greater. In some embodiments, the threshold is at least about 30% methylation in a target region of RAD9. In some embodiments, the threshold is between at least about 30% methylation and about 70% methylation in a target region of RAD9.

In some embodiments, the kit additionally comprises a plurality of reagents configured to effect DNA amplification and/or detection comprising dNTPs, recombinant thermostable DNA polymerase, and one or more PCR buffers. In some embodiments, the recombinant thermostable DNA polymerase includes but is not limited to Taq polymerase, Pfu DNA polymerase, KOD polymerase, GBD polymerase, a commercially available polymerases such as those sold by NEB, ThermoFischer and the like.

In some embodiments, the kit comprises a primer set comprising a plurality of forward and reverse primers designed to amplify one or more partially methylated forms of a DNA form of the RAD9 gene in which unmethylated cytosine residues present in DNA present in a blood, plasma, serum, or urine sample of the human subject. The sample can be any biological sample derived from a subject, which contains nucleic acids or polypeptides. Non-limiting examples of samples include blood, plasma, serum, or urine. The sample can be collected according to conventional techniques and used directly for analysis or stored. The sample can be treated prior to performing the method, in order to render or improve availability of nucleic acids for testing. Treatments include, for instance, lysis (e.g., mechanical, physical, or chemical), centrifugation. The nucleic acids can be pre-purified or enriched by conventional techniques, and/or reduced in complexity. Nucleic acids can also be treated with enzymes or other chemical or physical treatments to produce fragments thereof. In one embodiment, the sample is contacted with reagents, such as probes, primers, or ligands, in order to assess the methylated RAD9 nucleic acid. Contacting can be performed in any suitable device, such as a plate, tube, well, or glass. In some embodiments, the contacting is performed on a substrate coated with the reagent, such as an antibody or epitope binding fragment of an antibody. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids of the sample.

In another aspect, described herein is a nucleic acid primer comprising SEQ ID NO: 2. In some embodiments, the nucleic acid primer comprises a sequence is about 70%, about 75%, about 80%, about 85%, about 90%, about 93%, about 95%, about 97%, about 98%, or about 99% identical to SEQ ID NO: 2.

In another aspect, described herein is a nucleic acid primer comprising SEQ ID NO: 3, wherein the nucleic acid primer is biotinylated. In some embodiments, the nucleic acid primer comprises a sequence is about 70%, about 75%, about 80%, about 85%, about 90%, about 93%, about 95%, about 97%, about 98%, or about 99% identical to SEQ ID NO: 3.

In another aspect, described herein is a composition comprising a nucleic acid primer comprising SEQ ID NO: 2. In some embodiments, the composition further comprises a biotinylated nucleic acid primer comprising SEQ ID NO: 3.

In another aspect, described herein is a composition comprising a biotinylated nucleic acid primer comprising SEQ ID NO: 3.

In another aspect, described herein is a composition comprising a complex, wherein the complex comprises a modified RAD9 DNA molecule, wherein unmethylated cytosine residues present in DNA present in a urine sample from a subject have been modified; and a primer hybridized to the modified RAD9 DNA molecule. In some embodiments, the composition further comprises a recombinant thermostable DNA polymerase bound to the complex. In some embodiments, the primer further comprises one or more biotin molecules covalently attached to the primer. In some embodiments, the modified RAD9 DNA molecule has been amplified by PCR. In some embodiments, the primer comprises SEQ ID NO:2 or 3. In some embodiments, the primer comprises SEQ ID NO: 4 or 5. In some embodiments, the modified RAD9 DNA molecule comprises the transcription suppressor domain of intron 2. In some embodiments, the complex further comprises a second primer hybridized to the modified RAD9 DNA molecule. In some embodiments, the primer comprises SEQ ID NO: 2 and the second primer comprises SEQ ID NO: 3, or wherein the primer comprises SEQ ID NO: 3 and the second primer comprises SEQ ID NO: 2. In some embodiments, the primer comprises SEQ ID NO: 4 and the second primer comprises SEQ ID NO: 5, or wherein the primer comprises SEQ ID NO: 5 and the second primer comprises SEQ ID NO: 4. In some embodiments, the nucleic acid primer comprises a sequence is about 70%, about 75%, about 80%, about 85%, about 90%, about 93%, about 95%, about 97%, about 98%, or about 99% identical to SEQ ID NO: 4. In some embodiments, the nucleic acid primer comprises a sequence is about 70%, about 75%, about 80%, about 85%, about 90%, about 93%, about 95%, about 97%, about 98%, or about 99% identical to SEQ ID NO: 5. In some embodiments, the recombinant thermostable DNA polymerase includes but is not limited to Taq polymerase, Pfu DNA polymerase, KOD polymerase, GBD polymerase, a commercially available polymerases such as those sold by NEB, ThermoFischer and the like.

Methods for designing probes and/or primers for use in, for example, PCR or hybridization are known in the art. Probes and/or primers useful for detection can be assessed, for example, to determine those that do not form hairpins, self-prime or form primer dimers. Furthermore, a probe or primer (or the sequence thereof) is often assessed to determine the temperature at which it denatures from a target nucleic acid (i.e. the melting temperature of the probe or primer, or Tm). Methods for estimating Tm are known in the art. Methods for producing/synthesizing a probe or primer of the present invention are known in the art. For example, nucleic acid sequences encoding a molecule can be synthesized, in whole or in part, using chemical methods known in the art.

Methods of PCR are known in the art and described. Generally, for PCR two non-complementary nucleic acid primer molecules are hybridized to different strands of a nucleic acid template molecule at their respective annealing sites, and specific nucleic acid molecule copies of the template that intervene the annealing sites are amplified enzymatically.

The invention further provides for nucleic acid that are complementary to the converted one or more RAD9 regions under selective hybridization conditions. Complementary nucleic acids can selectively hybridize to the nucleic acid sequence under stringent hybridization conditions. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein. Non-limiting examples of stringent hybridization conditions include temperatures above 30° C., above 35° C., in excess of 42° C., and/or salinity of less than about 500 mM, or less than 200 mM. Hybridization conditions can be adjusted by the skilled artisan via modifying the temperature, salinity and/or the concentration of other reagents such as SDS or SSC. Preferably, the nucleic acids are designed to bind to the nucleic acid to which they are directed with a level of specificity which minimises the incidence of non-specific reactivity.

Labeling and conjugation techniques are known by those skilled in the art and can be used in various nucleic acid assays. Methods for producing labeled nucleic acids include, but are not limited to, oligolabeling, nick translation, end-labeling, or PCR amplification using a labeled nucleotide. Methods for producing biotin-labeled nucleic acids are known in the art.

### Detecting DNA Methylation and Sample Preparation

Any method for detecting DNA methylation can be used in the methods of the present invention. A number of methods are available for detection of differentially methylated DNA at specific loci in patient samples such as urine. For analysis of the proportion or extent of DNA methylation in a target gene, DNA is normally treated with sodium bisulfite and regions of interest amplified using primers and PCR conditions that will amplify independently of the methylation status of the DNA. The methylation of the overall amplicon or individual CpG sites can then be assessed by sequencing, including pyrosequencing, restriction enzyme digestion (COBRA) or by melting curve analysis. Alternative methods are also known in the art such as, but not limited to, ligation-based methods for analysis of methylation at specific CpG sites.

In one embodiment, the presence of one or more mutated nucleotides or the number of mutated sequences is determined by sequencing mutated DNA. One form of analysis comprises amplifying mutated nucleic acid using an amplification reaction described herein, for example, PCR. The amplified product is then directly sequenced or cloned and the cloned product sequenced. Methods for sequencing DNA are known in the art. As the treatment of nucleic acid with a compound, such as, for example, bisulfite results in non-methylated cytosines being mutated to uracil (and hence thymidine after an amplification process), analysis of the sequence determines the presence or absence of a methylated nucleotide. For example, by comparing the sequence obtained using a control sample or a sample that has not been treated with bisulfite, or the known nucleotide sequence of the region of interest with a treated sample facilitates the detection of differences in the nucleotide sequence. Any thymine residue detected at the site of a cytosine in the treated sample compared to a control or untreated sample may be considered to be caused by mutation as a result of bisulfite treatment.

In another embodiment, the presence of a mutated or non-mutated nucleotide in a bisulfite treated sample is detected using pyrosequencing. This method uses a primer that hybridizes to a site adjacent or close to the site of a cytosine that is methylated. Following hybridization of the primer and template in the presence of a DNA polymerase each of four modified deoxynucleotide triphosphates are added separately according to a predetermined dispensation order. Only an added nucleotide that is complementary to the bisulfite treated sample is incorporated and inorganic pyrophosphate (PPi) is liberated. The PPi then drives a reaction resulting in production of detectable levels of light. Such a method allows determination of the identity of a specific nucleotide adjacent to the site of hybridization of the primer.

Methods of solid phase pyrosequencing are known in the art and enable the high-throughput detection of methylation of a number of CpG dinucleotides. Other high throughput sequencing methods are encompassed by the present invention.

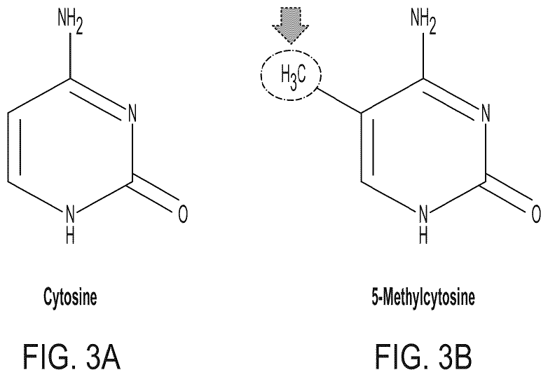

In some embodiments, the assay uses bisulfite pyrosequencing to quantify site-specific CpG methylation of a targeted region of RAD9. FIG. 3A shows cytosine and FIG. 3B shows 5-methylcytosine, the latter of which the assay detects. In some embodiments, the assay includes evaluation of total DNA, cell-free and/or cellular DNA from urine collected from men with advanced metastatic CRPC and aggressive-variant prostate cancer. In some embodiments, the results from the assay are compared to an age-matched cancer-free control cohort or a healthy subject that does not have cancer.

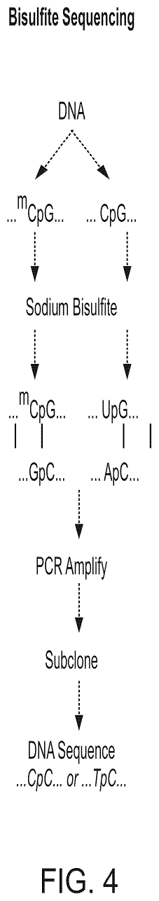

FIG. 4 is a flow chart showing the process of bisulfite sequencing. In some embodiments, RAD9 hypermethylation serves as a powerful non-invasive cancer diagnostic and prognostic tool, enabling reliable therapy response-based evaluation for men with aggressive and advanced disease. As shown in FIG. 4 which represents one embodiment, the collected DNA contains methylated and unmethylated CpG sites (^{m}CpG and CpG, respectively). The DNA is then treated with sodium bisulfite which converts each unmethylated cytosine into a uracil, while any methylated cytosine remains unaltered. The DNA is then subjected to PCR amplification and during this process each uracil is replaced with a thymine. In some embodiments, subclones are then created. The amplified DNA is then sequenced and occurrences of CpC sites indicate methylated cytosines while TpC sites indicate occurrences of unmethylated cytosine sites.

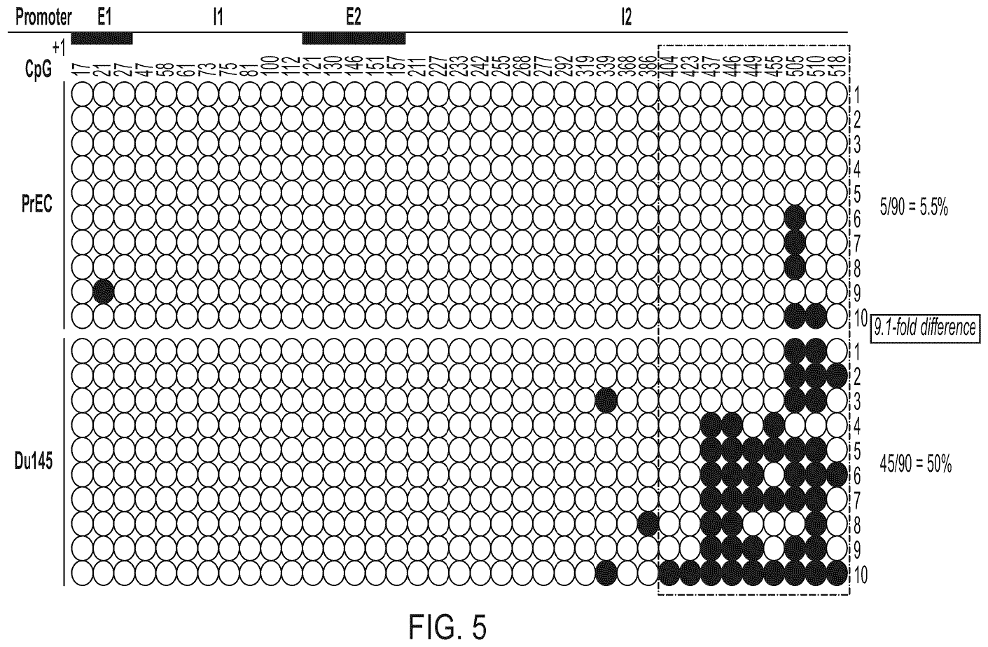

Referring to FIG. 5, DNA is collected from normal prostate epithelial cells (PrEC) and a prostate cancer cell line with neuroendocrine features (DU145), known to have low and excessively high RAD9 methylation, respectively. As can be seen in FIG. 5 from bisulfite sequencing results, the prostate cancer cell line with neuroendocrine features (DU145) has 9.1-fold higher methylation of CpG sites 404 to 518 (RAD9 (+1 ATG), see Table 1) within the transcription suppressor domain of RAD9 intron 2 compared to the normal PrECs. See Zhu, A., Zhang, X., and Lieberman, H. B. (2008) Rad9 has a functional role in human prostate carcinogenesis. Cancer Res. 68: 1267-1274, incorporated by reference in its entirety herein.

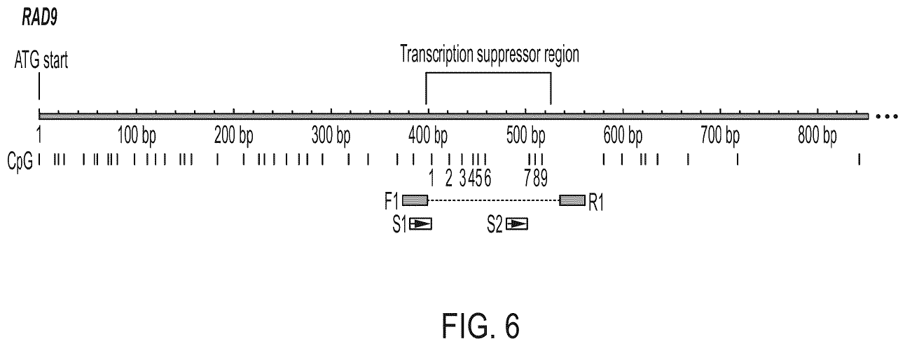

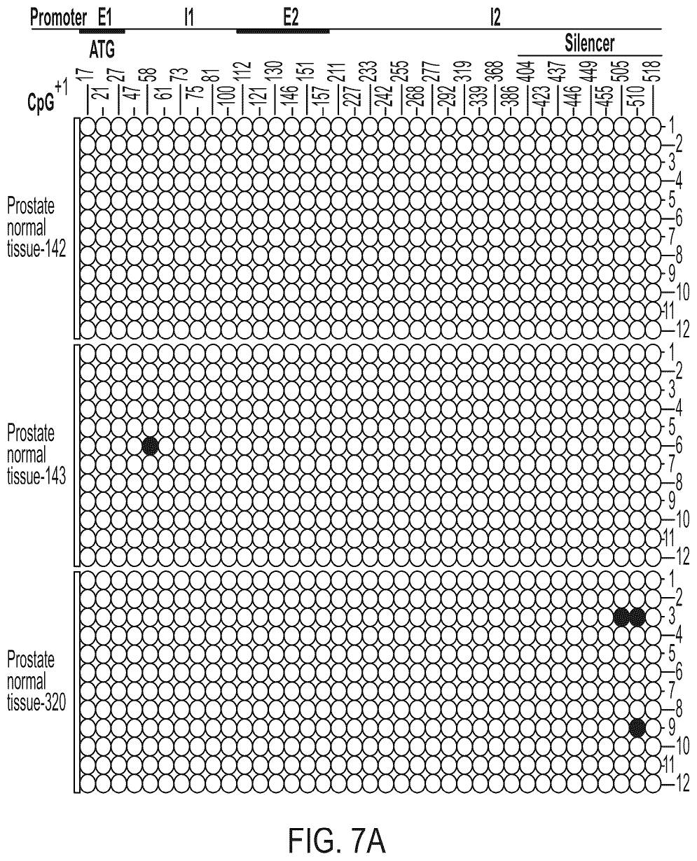

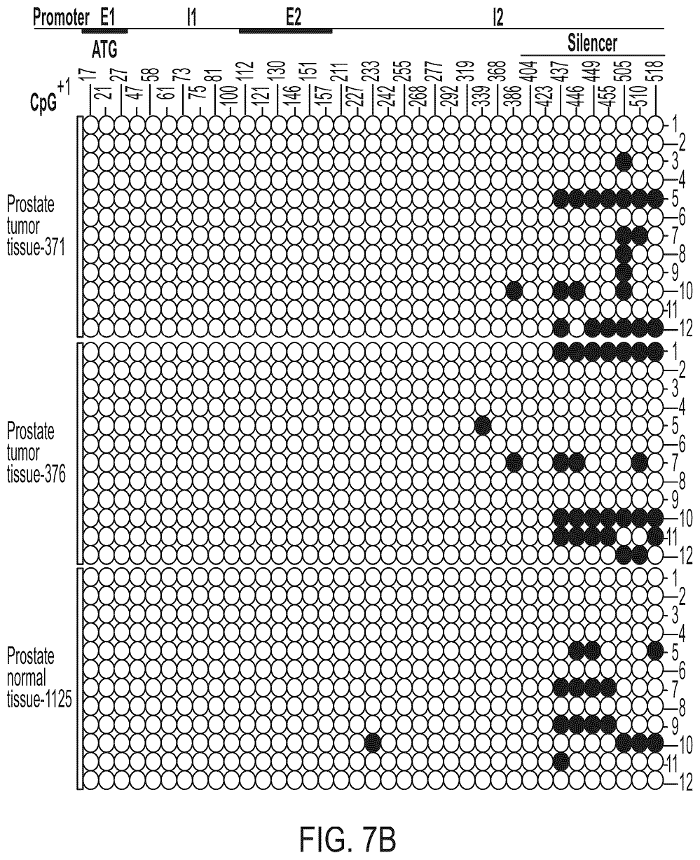

FIG. 6 shows the RAD9 gene with the suppressor regions targeted for detection of methylation of CpG sites in bisulfite sequencing. Referring to FIG. 7A-B, DNA is collected from normal human prostate tissue (FIG. 7A) and human prostate tumors (FIG. 7B). As can be seen in FIG. 7A-B from bisulfite sequencing results, the human prostate tumors (FIG. 7B) has a higher percentage of methylated CpG sites from 404 to 518 (RAD9 (+1 ATG), see Table 1) within the transcription suppressor (i.e., silencer) domain of RAD9 intron 2, compared to the normal human prostate tissue (FIG. 7A).

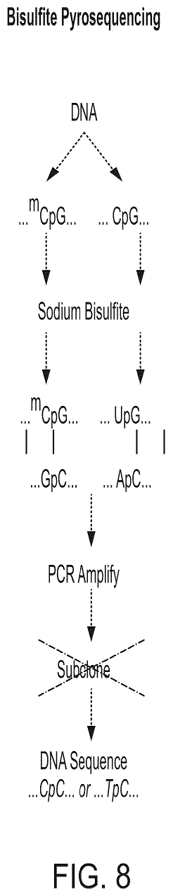

FIG. 8 shows one embodiment of bisulfite pyrosequencing that is similar to bisulfite sequencing described herein (see FIG. 5) but without the subcloning step. Bisulfite pyrosequencing is a DNA sequencing method based on the “sequencing by synthesis” principle, in which sequencing is performed by detecting the nucleotide incorporated by a DNA polymerase into bisulfite treated DNA. Pyrosequencing relies on light detection based on a chain reaction when pyrophosphate is released. In the pyrosequencing method, subcloning and sequencing individual clones are not necessary, thus improving efficiency (FIG. 8). Bisulfite pyrosequencing allows for determination of the percentage of cytosines methylated at individual sites in a population of DNA strands (C/T ratio). General bisulfite pyrosequencing methods are described in Weis et al., Reduced mRNA and protein expression of the genomic caretaker RAD9A in primary fibroblasts of individuals with childhood and independent second cancer. PLoS One 2011; 6(10):e25750; Galetzka, et al., Hypermethylation of RAD9A intron 2 in childhood cancer patients, leukemia and tumor cell lines suggest a role for oncogenic transformation. Research Square Posted 12 Aug. 2020; Galetzka et al., Hypermethylation of RAD9A intron 2 in childhood cancer patients, leukemia and tumor cell lines suggest a role for oncogenic transformation. EXCLI J 2022 Jan. 7; 21:117-143; Steven Bradburn, How Does Bisulfite Pyrosequencing Work?, Top Tip Bio, https://toptipbio.com/bisulfite-pyrosequencing/, the contents of all of which are incorporated by reference herein in their entireties.

In some embodiments, described herein are novel methods for preparing urine samples for bisulfite pyrosequencing. In some embodiments, the first step of a method for preparing urine samples for bisulfite pyrosequencing includes isolating genomic DNA (cellular and/or cell free) from urine. In some embodiments, the DNA is isolated from a urine sample that has a volume of between about 1 mL and 4 mL, between about 4 mL and 10 mL, between about 10 mL and 20 mL, between about 20 mL and 30 mL, between about 30 mL and 40 mL, or between about 40 mL and 50 mL. In some embodiments the urine sample has a volume of about 5 mL. In some embodiments, the urine sample has a volume of about 40 mL. In some embodiments, the DNA is isolated using a commercially available kit (e.g., Quick-DNA™ Urine Kit from Zymo Research). In some embodiments, the next step of the method involves performing bisulfite conversion of the isolated DNA. In some embodiments, the bisulfite conversion is performed using a commercially available kit (e.g., EZ DNA Methylation Kit from Zymo Research). In some embodiments, the next step of the method involves amplifying RAD9 from the bisulfite converted DNA using forward and reverse primers, wherein one of the primers is biotinylated. In some embodiments, a novel forward primer for PCR amplification is used having the nucleic acid sequence (from the 5′ to 3′end) TTTAGGGGGTGAAGTGTATTTAGAGAA (SEQ ID NO: 2). In some embodiments, a novel biotinylated reverse primer is used for PCR amplification having the nucleic acid sequence (from the 5′ to 3′end) ACAAACCCAACCCTCTAAC (SEQ ID NO: 3). In some embodiments, SEQ ID NO: 2 and SEQ ID NO: 3 are used in conjunction for PCR amplification. In some embodiments, commercially available polymerase chain reaction (PCR) enzyme/reagent kits are used for performing the amplification reaction (e.g., ZymoTaq PreMix). In some embodiments, after amplification, the next step of the method involves performing pyrosequencing using sequencing primers. In some embodiments, a novel sequencing primer is used having the nucleic acid sequence (from the 5′ to 3′end) GGTGAAGTGTATTTAGAGAAATT (SEQ ID NO: 4). In some embodiments, a novel sequencing primer is used having the nucleic acid sequence (from the 5′ to 3′end) GGAGGGTTTTTTAGTAGG (SEQ ID NO: 5). In some embodiments, SEQ ID NO: 4 and SEQ ID NO: 5 are used in conjunction for sequencing. In some embodiments, pyrosequencing is performed using commercially available automated pyrosequencing devices (e.g., Qiagen Pyromark Q48). In some embodiments, performing pyrosequencing includes the use of various reagents, buffers, and magnetic beads for nucleic acid separation as known in the art. In some embodiments, pyrosequencing is performed using the methodology described in the Pyromark® Q48 Autoprep User Manual (available at www.google.com/search?q=Pyromark % C2% AE+Q48+Autoprep+User+Manual&rlz=1C1GCE V_enUS977US977&oq=Pyromark % C2% AE+Q48+Autoprep+User+Manual&aqs=chrome..69i 57j0i22i30.1114j0j7&sourceid=chrome&ie=UTF-8), incorporated in its entirety herein.

In some embodiments, the novel methods described herein for preparing urine samples for bisulfite pyrosequencing result in more efficient and comprehensive detection of methylation of the RAD9 CpG target sites of interest (e.g., CpG sites 404-518).

The devices, kits, compositions, systems, and methods disclosed herein are not to be limited in scope to the specific embodiments described herein. Indeed, various modifications of the devices, kits, compositions, systems, and methods in addition to those described will become apparent to those of skill in the art from the foregoing description.

### EXAMPLES

### Example 1—Methylation of Cytosines at RAD9 CpG Sites for DU145 and PrEC Cell Lines

Table 2 shows the percent of methylated cytosines at 9 different CpG sites within the transcription suppressor domain of RAD9 intron 2 for PrEC and DU145 cell lines. Table 3 shows the average percent of methylation of cytosines across the 9 different CpG sites within the RAD9 transcription suppressor domain of RAD9 intron 2 for the PrEC and DU145 cell lines from Table 2. As shown in Table 3, there is a 10.1-fold increase in CpG site methylation in the prostate cancer cell line DU145 compared to the normal PrECs.

TABLE 2
Percent of methylation of cytosines at 9 RAD9 CpG sites

| | CpG Position | | | | | | | | |
|---|---|---|---|---|---|---|---|---|---|
| Cells | 1 | 2 | 3 | 4 | 5 | 6 | 7 | 8 | 9 |
| PrEc | 3 | 3 | 3 | 1 | 1 | 1 | 5 | 4 | 3 |
| DU145 | 6 | 9 | 36 | 34 | 31 | 19 | 45 | 37 | 27 |

TABLE 3
Average percent of methylation of cytosines at 9 RAD9 CpG sites

| | Average % of | |
|---|---|---|
| | 9 CpG Sites | DU145/PrEC |
| Cells | Methylated | CpG Methylation |
| PrEC | 2.67% | |
| | | 10.1 X |
| DU145 | 27.1% | |

### Example 2—Methylation of Cytosines at RAD9 CpG Sites in Human Participants

Two healthy controls and two patients with prostate cancer were studied. Participants 1 and 2 were healthy controls while Participants 3 and 4 were prostate cancer patients. Patient 3 was a 51-year-old man with locally advanced, hormone resistant prostate cancer who initially presented with a PSA measuring 666 ng/mL in August of 2021 in the setting of grade group 5 disease and 6/12 prostate biopsy cores and pelvic lymphadenopathy (N+). He was started on hormonal therapy in October of 2021. On Feb. 2, 2022, his PSA measured 144 ng/mL (normal being <4 ng/mL) four months after starting hormonal therapy. Participant 4 was a 72-year-old man with very high risk prostate cancer, 15/16 cores, grade group 5, PSA 5.6 (low, possibly suggestive of aggressive-variant), and was treatment naive. Urine from Participant 4 was split into 2 aliquots (4a, 4b), then independently processed and evaluated. The urine samples from all patients were prepared for pyrosequencing to determine methylation at CpG sites of interest in RAD9 using the novel pyrosequencing preparation methods described herein.

Table 4 shows the percent of methylated cytosines at 9 different CpG sites within the transcription suppressor domain of RAD9 intron 2 for healthy Participant 1 in cellular DNA collected from urine (“#1 C”) and in cell-free DNA collected from urine (“#1 CF”).

TABLE 4
Percent of methylation of cytosines at 9 RAD9 CpG
sites for healthy Participant 1

| | CpG Position | | | | | | | | |
|---|---|---|---|---|---|---|---|---|---|
| Participant | 1 | 2 | 3 | 4 | 5 | 6 | 7 | 8 | 9 |
| #1 C | 5 | 7 | 16 | 15 | 8 | 8 | 43 | 33 | 14 |
| #1 CF | 6 | 6 | 15 | 12 | 7 | 7 | 47 | 34 | 12 |

Table 5 shows the percent of methylated cytosines at 9 different CpG sites within the transcription suppressor domain of RAD9 intron 2 for healthy Participant 2 in cellular DNA collected from urine (“#2 C”) and in cell-free DNA collected from urine (“#2 CF”).

TABLE 5
Percent of methylation of cytosines at 9 RAD9 CpG
sites for healthy Participant 2

| | CpG Position | | | | | | | | |
|---|---|---|---|---|---|---|---|---|---|
| Participant | 1 | 2 | 3 | 4 | 5 | 6 | 7 | 8 | 9 |
| #2 C | 3 | 3 | 4 | 3 | 3 | 2 | 15 | 9 | 4 |
| #2 CF | 8 | 11 | 23 | 24 | 17 | 14 | 38 | 33 | 21 |

Table 6 shows the percent of methylated cytosines at 9 different CpG sites within the transcription suppressor domain of RAD9 intron 2 for cancer Participant 3 in cellular DNA collected from urine (“#3 C”) and in cell-free DNA collected from urine (“#3 CF”).

TABLE 6
Percent of methylation of cytosines at 9 RAD9
CpG sites for cancer Participant 3

| | CpG Position | | | | | | | | |
|---|---|---|---|---|---|---|---|---|---|
| Participant | 1 | 2 | 3 | 4 | 5 | 6 | 7 | 8 | 9 |
| #3-C | 2 | 2 | 4 | 3 | 3 | 3 | 12 | 9 | 3 |
| 3-CF | 4 | 6 | 4 | 6 | 4 | 3 | 12 | 8 | 3 |

Table 7 shows the percent of methylated cytosines at 9 different CpG sites within the transcription suppressor domain of RAD9 intron 2 for cancer Participant 4 in cellular DNA collected from urine (“#4 C”) and in cell-free DNA collected from urine that was split into 2 aliquots (“#4 CFa” and “#4 CFb”).

TABLE 7
Percent of methylation of cytosines at 9 RAD9 CpG
sites for cancer Participant 4

| | CpG Position | | | | | | | | |
|---|---|---|---|---|---|---|---|---|---|
| Participant | 1 | 2 | 3 | 4 | 5 | 6 | 7 | 8 | 9 |
| #4-C | 4 | 5 | 6 | 9 | 4 | 3 | 29 | 18 | 8 |
| #4-CFa | 39 | 27 | 30 | 36 | 30 | 34 | 36 | 38 | 39 |
| #4-CFb | 67 | 70 | 66 | 72 | 71 | 73 | 68 | 64 | 73 |

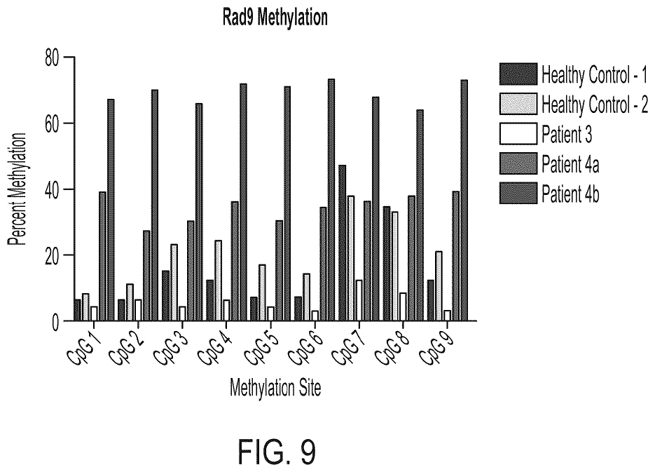

FIG. 9 summarizes Tables 3-6 above and shows the percent methylation at 9 CpG sites of RAD9 in the cell-free (“CF”) fraction of human urine for the two healthy controls (Healthy Control 1 and Healthy Control 2) and two prostate cancer patients (Patient 3 and 4; Patient 4's sample was split into two aliquots: 4a and 4b).

### Example 3—Validation of Urine-Based Diagnostic Assay

In some embodiments, the disclosure is directed to a urine-based diagnostic assay for the detection and quantification of RAD9 methylation status in human urine. In some embodiments, the urine-based diagnostic assay is validated by identifying a cohort of 11 patients with high volume, metastatic castrate resistant prostate cancer (mCRPC), 11 patients with aggressive-variant prostate cancer, and 11 patients with hormone sensitive prostate adenocarcinoma, along with 33 age-matched cancer free controls. From these patients, fresh urine specimens are collected for bisulfite pyrosequencing analysis. In some embodiments, the urine samples are prepared for pyrosequencing to determine methylation at CpG sites of interest in RAD9 using the novel pyrosequencing preparation methods described herein. In some embodiments, the efficacy of the disclosed RAD9 methylation assay to detect disease progression is improved compared to standard imaging and PSA, in men with metastatic CRPC and aggressive-variant prostate cancer.

In some embodiments, the bisulfite pyrosequencing assay for cellular and cell free DNA in urine is optimized. In some embodiments, urine samples from prostate cancer patients and matched cancer-free controls are accrued and analyzed. In some embodiments, specimen handling for specific biomarkers is optimized and biomarkers are validated.

### Cohort Features

### High Volume Prostate Adenocarcinoma

Men with a diagnosis of prostate cancer (ICD10 C61).
Men with metastatic prostate cancer (most common=bone metastases).
Men with PSA>50 ng/mL.
Men with normal chromogranin A<103 ng/mL.
Men seen in a Columbia University clinic in the last 6 months.
Men without other cancer diagnosis.
Men on or off cancer-directed therapy.

### Neuroendocrine Prostate Cancer

Men with a diagnosis of prostate cancer (ICD10 C61).
Men with elevated chromogranin A >103 ng/mL (in blood, not required).
Men seen in a Columbia University clinic in the last 6 months.
Men without other cancer diagnosis.
Men on or off cancer-directed therapy.
All PSA (low or high) are included, but low <4 ng/mL is of particular interest.
Urine Processing, Bisulfite Treatment, and Primer Design.

Matched cancer-free controls are also included.

### Example 4—Urine Processing, Bisulfite Treatment, and Primer Design Urine Processing

DNA Yield—DNA yield may vary depending on the urine itself. Female urine typically yields more DNA than male urine. Urine DNA from a healthy female individual ranges on average from about 6 to about 1000 ng/ml. DNA from a healthy male individual ranges on average from about 2 to about 20 ng/ml. Total DNA, cellular, or cell-free is isolated. The DNA sizes capable of being recovered for the assay are DNA fragments from about 100 bp to about 23 kb.

### Bisulfite Treatment

DNA Input—Samples containing 500 μg to 2 μg of DNA. In some embodiments, for optimal results, the amount of input DNA is from about 200 to about 500 ng.

Conversion Efficiency—99% of non-methylated C residues are converted to U; >99% protection of methylated cytosines.

1.-26. (canceled)

27. A method of treating prostate, bladder, or kidney cancer in a subject comprising: (a) measuring methylation status at a plurality of CpG sites within a region of a RAD9 gene in DNA present in a urine sample from the subject; and (b) administering an effective amount of a prostate, bladder, or kidney cancer treatment to the subject who has been determined to have an increased level of methylation at the plurality of CpG sites, thereby treating prostate, bladder, or kidney cancer in the subject.

28. The method of claim 27, wherein the subject is a human.

29. The method of claim 28, wherein measuring the amount of the methylation comprises performing bisulfite sequencing or bisulfite pyrosequencing.

30. The method of claim 28, wherein the methylation status of the plurality of CpG sites within a region of a RAD9 gene is determined in cell-free DNA, cellular DNA, or total DNA present in the urine sample.

31. The method of claim 27, wherein DNA present in the urine sample from the subject is isolated prior to measuring the methylation status of the plurality of CpG sites.

32. The method of claim 31, wherein the isolated DNA comprises an amount of cell-free DNA from the urine sample.

33. The method of claim 32, wherein the amount of cell-free DNA isolated from the urine sample is about 500 μg to about 2 μg.

34. The method of claim 33, wherein the amount of cell-free DNA isolated from the urine sample is about 200 ng to about 500 ng.

35. The method of claim 31, wherein the isolated DNA comprises an amount of cellular DNA from the urine sample.

36. The method of claim 35, wherein the amount of cellular DNA isolated from the urine sample is about 500 μg to about 2 μg.

37. The method of claim 36, wherein the amount of cellular DNA isolated from the urine sample is about 200 ng to about 500 ng.

38. The method of claim 28, wherein the region of the RAD9 gene comprises a transcription suppressor domain of intron 2.

39. The method of claim 38, wherein the plurality of CpG sites comprises sites 404 to 518 within intron 2.

40. The method of claim 27, wherein the subject is determined to have an increased level of methylation at the plurality of CpG sites if the amount of methylation of the plurality of CpG sites within the region of the RAD9 gene in DNA present in the urine sample from the subject is at least about 1.5 times greater methylation compared to an amount of methylation at a plurality of the same CpG sites in the same region of the RAD9 gene in DNA present in a second urine sample obtained from a healthy subject that does not have cancer or in a cohort of urine samples obtained from a plurality of healthy subjects that do not have cancer.

41. The method of claim 28, wherein the prostate, bladder or kidney cancer treatment is surgery, transurethral resection of bladder tumor (TURBT), radiation therapy, Lutetium-PSMA, radium-223 (Xofigo), cryotherapy, high-intensity focused ultrasound (HIFU), focal laser ablation, chemotherapy, immunotherapy, hormonal therapy, orchiectomy, administration of antineoplastic agents, or a combination thereof.

42. The method of claim 41, wherein the immunotherapy comprises administering a checkpoint inhibitor, a bi-specific antibody, an antibody-drug conjugate, a cytokine, or performing an intravesical immunotherapy.

43. The method of claim 42, wherein the cytokine is IL-2.

44. The method of claim 42, wherein the intravesical immunotherapy is Bacillus Calmette-Guerin (BSG).

45. The method of claim 41, wherein the administration of antineoplastic agents comprises administering a PARP inhibitor.

46.-77. (canceled)

78. The method of claim 27, wherein measuring the methylation status at a plurality of CpG sites within a region of a RAD9 gene comprises amplification of one or more partially methylated forms of a DNA form of the RAD9 gene with a primer set comprising SEQ ID NO: 2 or SEQ ID NO: 3.

79. The method of claim 27, wherein measuring the methylation status at a plurality of CpG sites within a region of a RAD9 gene comprises sequencing the RAD9 region with primers comprising SEQ ID NO: 4 or SEQ ID NO: 5.
